(19) United States
(12) Patent Application Publication
(10) Pub. No.: US 2021/0189473 A1
(43) Pub. Date: Jun. 24, 2021

(54) UNIVERSAL TAIL PRIMERS WITH MULTIPLE BINDING MOTIFS FOR MULTIPLEXED DETECTION OF SINGLE NUCLEOTIDE POLYMORPHISMS

(71) Applicant: ChromaCode,Inc., Carlsbad, CA (US)

(72) Inventors: Aditya RAJAGOPAL, Carlsbad, CA (US); Lucien JACKY, Carlsbad, CA (US); Karen L. MENGE, Carlsbad, CA (US)

(21) Appl. No.: 17/273,935

(22) PCT Filed: Sep. 6, 2018
(86) PCT No.: PCT/US2019/050050

§ 371 Date: Mar. 5, 2021

Related U.S. Application Data
(60) Provisional application No. 62/829,474, filed on Apr. 4, 2019.
(60) Provisional application No. 62/728,262, filed on Sep. 7, 2018.

(51) Int. Cl. C12Q 1/6827; C12Q 1/6851; C12Q 1/6825

(52) U.S. Cl. CPC C12Q 1/6827; C12Q 2600/156; C12Q 1/6825; C12Q 1/6851

(57) ABSTRACT

The present disclosure provides methods and compositions for identifying and discriminating between one or more target nucleic acids within a sample. The disclosed methods may be useful for detecting single nucleotide polymorphisms (SNPs). The disclosed methods may be useful in identifying or detecting the presence or absence of a nucleotide mutation. The use of mutation-specific oligonucleotide probes with universal tails enables specific detection of a SNP target present in a sample.

### CROSS-REFERENCE

This application claims the benefit of U.S. Provisional Patent Application No. 62/728,262, filed on Sep. 7, 2018; and U.S. Provisional Patent Application No. 62/829,474 filed on Apr. 4, 2019—each of which are incorporated by reference in their entirety for all purposes.

### BACKGROUND

Real-time PCR (qPCR) is a broadly used method for the detection of single nucleotide polymorphisms (SNPs). A SNP is a polymorphism of a single nucleotide that occurs at a specific position in the genome. Because single-base mutations may consequently increase or decrease an individual's susceptibility to a wide range of diseases, it is advantageous to know whether an individual possess a known SNP mutation. The use of labeled oligonucleotide probes specific for SNP mutations enables specific detection of a SNP target present in a sample.

### SUMMARY

Disclosed herein, in some aspects, is a method for determining the presence or absence of a target nucleic acid in the presence of a non-target nucleic acid, said method comprising: (A) providing a sample comprising, or potentially comprising, said target nucleic acid and said non-target nucleic acid; (B) forming a mixture comprising: (i) said sample; (ii) a forward primer comprising a first region configured to hybridize to said target nucleic acid under amplification conditions and configured to not hybridize to said non-target nucleic acid under said amplification conditions, and a second region configured to not hybridize to said target nucleic acid under said amplification conditions; and (iii) a signal generating nucleic acid probe, where said signal generating nucleic acid probe anneals to said second region, or regions complementary thereto, when subjected to said amplification conditions; (C) subjecting said mixture to said amplification conditions, said amplification conditions appropriate to amplify said target nucleic acid with an amplification reaction, thereby amplifying said target nucleic acid such that said signal generating nucleic acid probe is degraded and a signal is generated if said target nucleic acid is present in said mixture; and (D) detecting the presence or absence of said signal, thereby determining the presence or absence of said target nucleic acid in the presence of said non-target nucleic acid. In some embodiments, said target nucleic acid is an allele. In some embodiments, said target nucleic acid is a mutant sequence and said non-target nucleic is a wild type sequence. In some embodiments, the sequence of said target nucleic acid and the sequence of said non-target nucleic acid is different at least one nucleotide. In some embodiments, the sequence of said target nucleic acid and the sequence of said non-target nucleic acid is different by only one nucleotide. In some embodiments, the sequence of said target nucleic acid and the sequence of said non target nucleic acid is different by no more than five nucleotides.

In some embodiments, said forward primer is specific to and selectively amplifies only said target nucleic acid. In some embodiments, said mixture does not employ the use of a nucleic acid or peptide blocking agents. In some embodiments, said mixture further comprising a reverse primer. In some embodiments, said forward primer is configured to hybridize to a first area of a nucleic acid sequence of said target nucleic acid, and said reverse primer is configured to hybridize to a second area of the nucleic acid sequence of said target nucleic acid, thereby being configured to amplify the nucleic acid sequence under conditions sufficient for nucleic acid amplification. In some embodiments, said first area comprises a 3′ end of said nucleic acid sequence. In some embodiments, said second area comprises a 5′ end of said nucleic acid sequence. In some embodiments, said reverse primer comprises a sequence with complementarity or is homologous to a sequence on both target nucleic acid and non-target nucleic acid. In some embodiments, said reverse primer is a locus specific primer. In some embodiments, said reverse primer is a universal primer.

In some embodiments, said reverse primer is configured such that upon said thermally cycling, said signal generating nucleic acid probe is digested. In some embodiments, said mixture further comprises a nucleic acid enzyme. In some embodiments, said amplification reaction is a polymerase chain reaction (PCR). In some embodiments, said PCR is a quantitative polymerase chain reaction (qPCR). In some embodiments, said amplification conditions comprises: a dNTP, a salt, a buffer, or a combination thereof.

In some embodiments, said signal generating nucleic acid probe further comprises a signal tag. In some embodiments, said signal tag generates said signal. In some embodiments, said signal tag generates said signal upon degradation of said cleavable, signal generating nucleic acid probe by the 5′ to 3′ exonuclease activity of said nucleic acid enzyme, thereby liberating said signal for detection by a real time PCR instrument.

In some embodiments, said amplification conditions comprise thermal cycling and each thermal cycle is performed at an annealing temperature appropriate for annealing said forward primer to said target nucleic acid. In some embodiments, said second region comprises: (A) a target-specific tail segment; and (B) a universal tail segment. In some embodiments, said first region is positioned on the 3′ end of forward primer. In some embodiments, said universal tail segment is located on the 5′ end of said forward primer. In some embodiments, said target-specific tail segment is flanked on the 3′ by said target-specific segment, and flanked on the 5′ end by said universal tail segment on said forward primer.

In some embodiments, a plurality of forward primers may be used. In some embodiments, each second region of said plurality of forward primers comprises identical nucleotide sequences. In some embodiments, each second region of said plurality of forward primers comprises dissimilar nucleotide sequences. In some embodiments, each second region of said plurality of forward primers comprises unique nucleotide sequences.

In some embodiments, said signal generating nucleic acid probe comprises sequence complementary or homologous to said forward primer. In some embodiments, said signal generating nucleic acid probe is a target-specific probe. In some embodiments, said signal generating nucleic acid probe is a target-specific probe which comprises (A) a sequence complementary or homologous to said 3′ end of said universal tail, (B) a sequence complementary or homologous to said entire target-specific tail, and (C) a sequence complementary or homologous to a portion of said first region. In some embodiments, said signal generating nucleic acid probe is a target-specific probe which comprise: (A) a sequence complementary or homologous to said 3′ end of said universal tail, and (B) a sequence complementary or homologous to a portion of the target-specific tail. In some embodiments, said nucleic acid probe binds to said target nucleic acid in an amplicon generated by second strand synthesis initiated in said amplification reaction.

In some embodiments, said mixture further comprises a second nucleic acid primer. In some embodiments, said second nucleic acid primer is a target specific primer. In some embodiments, said second nucleic acid primer is configured to hybridize with said target nucleic acid or a derivative thereof and is not configured to hybridize with said non-target nucleic or derivative thereof. In some embodiments, the sequence of said target nucleic acid and the sequence of said non-target nucleic acid are different by at least one nucleotide. In some embodiments, the sequence of said target nucleic acid and the sequence of said non-target nucleic acid are different by only one nucleotide. In some embodiments, the sequence of said target nucleic acid and the sequence of said non-target nucleic acid are different by no more than five nucleotides. In some embodiments, said target specific primer is homologous or complementary to a sequence of said target nucleic acid. In some embodiments, said second nucleic acid primer is a universal primer. In some embodiments, said universal primer is complementary or homologous to a sequence of said second region. In some embodiments, said second nucleic acid primer is configured to digest said cleavable, signal generating probe upon thermocycling. In some embodiments, said signal generating probe comprises a sequence homologous or complementary to said target nucleic acid.

In some embodiments, said signal generating probe is configured to anneal to said second region and said first region. In some embodiments, said signal generating nucleic acid probe is configured to hybridize to said second region or said first region. In some embodiments, said signal generating nucleic acid probe is configured to hybridize to only said first region. In some embodiments, said signal generating nucleic acid probe is configured to hybridize to only said second region. In some embodiments, said signal generating nucleic acid probe is configured to hybridize to said target-specific tail segment. In some embodiments, said signal generating nucleic acid probe is configured to hybridize to said target-specific tail segment and not hybridize to a sequence, or complement thereof, of said target nucleic acid.

In some embodiments, said target nucleic acid and said non-target a nucleic acid are different at a divergence location comprising at least one nucleotide, and said signal generating nucleic acid probe is configured to hybridize to said target-specific tail segment and not hybridize to said divergence location comprising at least one nucleotide, or complement thereof. In some embodiments, said second region comprises: (A) a universal probe binding motif, and (B) a universal primer binding motif. In some embodiments, said signal generating probe is configured to bind said universal probe binding motif. In some embodiments, said mixture further comprises a universal primer configured to bind said universal primer binding motif. In some embodiments, said target-specific segment is positioned on the 3′ end of said forward primer. In some embodiments, said universal primer binding motif is located on the 5′ end of said universal tail primer. In some embodiments, said universal probe binding motif is flanked by the 5′ end of said first region, and the 3′ end of said universal primer binding motif.

In some embodiments, each universal primer binding motif comprises identical nucleotide sequences when said plurality of forward primers are used. In some embodiments, each universal primer binding motif comprises dissimilar nucleotide sequences when said plurality of forward primers are used.

In some embodiments, said second region comprises a second universal probe binding motif. In some embodiments, said mixture further comprises a probe with a sequence complementary or homologous to said second universal probe binding motif. In some embodiments, said universal probe binding motif and said second universal probe binding motif comprise the same sequence. In some embodiments, said universal probe binding motif and said second universal probe binding motif comprise a different sequence. In some embodiments, said universal probe binding motif and said second universal probe binding motif are configured to bind the same probe sequence. In some embodiments, said universal probe binding motif and said second universal probe binding motif are configured to bind a different probe sequence. In some embodiments, said second universal probe binding motif is adjacent to said universal binding motif and said second universal probe binding motif and said universal binding motif are on the 3′ end of said first region and are on the 5′ of said universal primer binding motif. In some embodiments, each universal primer binding motif comprises identical nucleotide sequences when a plurality of said forward primers are used. In some embodiments, each universal primer binding motif comprises dissimilar nucleotide sequences when said plurality of said forward primers are used. In some embodiments, each of said at least two universal probe binding motifs comprises a unique nucleotide sequence when said plurality of forward primers are used.

Disclosed herein, in some aspects, is a method for determining the presence or absence of a target nucleic acid in the presence of a non-target nucleic acid, said method comprising: (A) providing a sample comprising said target nucleic acid; (B) forming a mixture comprising: (i) said sample; (ii) a non-extendible nucleic acid primer, where said non-extendible nucleic acid primer comprises a first region configured to hybridize to said target nucleic acid and configured not to bind to said non-target nucleic acid, and a second region configured to not hybridize to said target nucleic acid, and where said non-extendible nucleic acid primer comprises a target-specific RNA base, DNA base, or unnatural base, configured to, when said first region is hybridized to said target nucleic acid, form a base hybrid pair at a target base of said target nucleic acid and not form said base hybrid pair at a corresponding base in the non-target nucleic acid; and (iii) a signal generating nucleic acid probe; (C) subjecting said mixture to cleavage conditions such that nucleic acids comprising said base hybrid pair are cleaved such that said non-extendible nucleic acid is cleaved, thereby converting said non-extendible nucleic acid primer to an extendible primer; (D) subjecting said mixture to said amplification conditions, said amplification conditions appropriate to amplify said target nucleic acid with an amplification reaction, such that said signal generating nucleic acid probe is degraded and a signal is generated if said target nucleic acid is present in said mixture; and (E) detecting the presence or absence of said signal, thereby determining the presence or absence of said target nucleic acid in the presence of said non-target nucleic acid.

In some embodiments, said amplification reaction comprises a reverse transcriptase polymerase chain reaction (RT-PCR) or a polymerase chain reaction (PCR). In some embodiments, said target-specific RNA base, DNA base, or unnatural base of said non-extendible nucleic acid primer is at a location complementary to said target nucleic acid. In some embodiments, said target-specific RNA base, DNA base, or unnatural base facilitates the selective amplification of said target nucleic acid. In some embodiments, said base hybrid pair is a DNA: RNA pair, a DNA:unnatural base, or an RNA:unnatural base pair.

In some embodiments, said non-extendible nucleic acid primer is cleaved at a location adjacent to or at said base hybrid pair as described in (C). In some embodiments, said cleavage conditions comprise an additional reagent or enzyme for cleaving said first non-extendible nucleic acid primer. In some embodiments, said additional enzyme is ribonuclease H.

In some embodiments, said target nucleic acid is an allele. In some embodiments, said target nucleic acid is a mutant sequence and said non-target nucleic is a wild type sequence. In some embodiments, the sequence of said target nucleic acid and the sequence of said non-target nucleic acid is different at least one nucleotide. In some embodiments, the sequence of said target nucleic acid and the sequence of said non-target nucleic acid is different by only one nucleotide. In some embodiments, the sequence of said target nucleic acid and the sequence of said non target nucleic acid is different by no more than five nucleotides.

In some embodiments, said extendible primer is specific to and selectively amplifies only said target nucleic acid. In some embodiments, said mixture does not employ the use of a nucleic acid or peptide blocking agents. In some embodiments, said mixture further comprising a reverse primer. In some embodiments, said extendible primer is configured to hybridize to a first area of a nucleic acid sequence of said target nucleic acid, and said reverse primer is configured to hybridize to a second area of the nucleic acid sequence of said target nucleic acid, thereby being configured to amplify the nucleic acid sequence under conditions sufficient for nucleic acid amplification. In some embodiments, said first area comprises a 3′ end of said nucleic acid sequence. In some embodiments, said second area comprises a 5′ end of said nucleic acid sequence. In some embodiments, said reverse primer comprises a sequence with complementarity or is homologous to a sequence on both target nucleic acid and non-target nucleic acid. In some embodiments, said reverse primer is a locus specific primer. In some embodiments, said reverse primer is a universal primer.

In some embodiments, said reverse primer is configured such that upon said thermally cycling said signal generating nucleic acid probe is digested. In some embodiments, said mixture further comprises a nucleic acid enzyme. In some embodiments, said amplification reaction is a polymerase chain reaction (PCR). In some embodiments, said PCR is a quantitative polymerase chain reaction (qPCR). In some embodiments, said amplification conditions comprises: a dNTP, a salt, a buffer, or a combination thereof.

In some embodiments, said signal generating nucleic acid probe further comprises a signal tag. In some embodiments, said signal tag generates said signal. In some embodiments, said signal tag generates said signal upon degradation of said cleavable, signal generating nucleic acid probe by the 5′ to 3′ exonuclease activity of said nucleic acid enzyme, thereby liberating said signal for detection by a real time PCR instrument.

In some embodiments, said amplification conditions comprise thermal cycling and each thermal cycle is performed at an annealing temperature appropriate for annealing said extendible primer to said target nucleic acid. In some embodiments, said second region comprises: (A) a target-specific tail segment; and (B) a universal tail segment. In some embodiments, said first region is positioned on the 3′ end of extendible primer. In some embodiments, said universal tail segment is located on the 5′ end of said extendible primer. In some embodiments, said target-specific tail segment is flanked on the 3′ by said target-specific segment, and flanked on the 5′ end by said universal tail segment on said non-extendible primer.

In some embodiments, a plurality of non-extendible primers is used. In some embodiments, each second region of said plurality of non-extendible primers comprises identical nucleotide sequences. In some embodiments, each second region of said plurality of non-extendible primers comprises dissimilar nucleotide sequences. In some embodiments, each second region of said plurality of non-extendible primers comprises unique nucleotide sequences.

In some embodiments, said signal generating nucleic acid probe comprises sequence complementary or homologous to said non-extendible primer. In some embodiments, said signal generating nucleic acid probe is a target-specific probe. In some embodiments, said signal generating nucleic acid probe is a target-specific probe which comprises (A) a sequence complementary or homologous to said 3′ end of said universal tail, (B) a sequence complementary or homologous to said entire target-specific tail, and (C) a sequence complementary or homologous to a portion of said first region. In some embodiments, said signal generating nucleic acid probe is a target-specific probe which comprise: (A) a sequence complementary or homologous to said 3′ end of said universal tail, and (B) a sequence complementary or homologous to a portion of the target-specific tail. In some embodiments, said nucleic acid probe binds to said target nucleic acid in an amplicon generated by second strand synthesis initiated in said amplification reaction.

In some embodiments, said mixture further comprises a second nucleic acid primer. In some embodiments, said second nucleic acid primer is a target specific primer. In some embodiments, said second nucleic acid primer is configured to hybridize with said target nucleic acid or a derivative thereof and not to hybridize with said non-target nucleic or derivative thereof. In some embodiments, the sequence of said target nucleic acid and the sequence of said non-target nucleic acid are different at least one nucleotide. In some embodiments, the sequence of said target nucleic acid and the sequence of said non-target nucleic acid are different by only one nucleotide. In some embodiments, said the sequence of said target nucleic acid and the sequence of said non-target nucleic acid are different by no more than five nucleotides. In some embodiments, said target specific primer is homologous or complementary to a sequence of said target nucleic acid. In some embodiments, said second nucleic acid primer is a universal primer. In some embodiments, said universal primer is complementary or homologous to a sequence of said second region. In some embodiments, said second nucleic acid primer is configured to digest said cleavable, signal generating probe upon thermocycling. In some embodiments, said signal generating probe comprises a sequence homologous or complementary to said target nucleic acid.

In some embodiments, said signal generating probe is configured to anneal to said second region and said first region. In some embodiments, said signal generating nucleic acid probe is configured to hybridize to said second region or said first region. In some embodiments, said signal generating nucleic acid probe is configured to hybridize to only said first region. In some embodiments, said signal generating nucleic acid probe is configured to hybridize to only said second region. In some embodiments, said signal generating nucleic acid probe is configured to hybridize to said target-specific tail segment. In some embodiments, said signal generating nucleic acid probe is configured to hybridize to said target-specific tail segment and not hybridize to a sequence, or complement thereof, of said target nucleic acid.

In some embodiments, said target nucleic acid and said non-target a nucleic acid are different at a divergence location comprising at least one nucleotide, and said signal generating nucleic acid probe is configured to hybridize to said target-specific tail segment and not hybridize to said divergence location comprising at least one nucleotide, or complement thereof. In some embodiments, said second region comprises: (A) a universal probe binding motif; and (B) a universal primer binding motif. In some embodiments, said signal generating probe is configured to bind said universal probe binding motif. In some embodiments, said mixture further comprise a universal primer configured to bind said universal primer binding motif. In some embodiments, said target-specific segment is positioned on the 3′ end of said non-extendible primer. In some embodiments, said universal primer binding motif is located on the 5′ end of said universal tail primer. In some embodiments, said universal probe binding motif is flanked by the 5′ end of said first region, and the 3′ end of said universal primer binding motif.

In some embodiments, each universal primer binding motif comprises identical nucleotide sequences when said plurality of non-extendible primers are used. In some embodiments, each universal primer binding motif comprises dissimilar nucleotide sequences when said plurality of non-extendible primers are used.

In some embodiments, said second region comprises a second universal probe binding motif. In some embodiments, said mixture further comprises a probe with a sequence complementary or homologous to said second universal probe binding motif. In some embodiments, said universal probe binding motif and said second universal probe binding motif comprise the same sequence. In some embodiments, said universal probe binding motif and said second universal probe binding motif comprise a different sequence. In some embodiments, said universal probe binding motif and said second universal probe binding motif are configured to bind the same probe sequence. In some embodiments, said universal probe binding motif and said second universal probe binding motif are configured to bind a different probe sequence. In some embodiments, said second universal probe binding motif is adjacent to said universal binding motif and said second universal probe binding motif and said universal binding motif are on the 3′ end of said first region and are on the 5′ of said universal primer binding motif. In some embodiments, each universal primer binding motif comprises identical nucleotide sequences when a plurality of said non-extendible primers are used. In some embodiments, each universal primer binding motif comprises dissimilar nucleotide sequences when said plurality of said non-extendible primers are used. In some embodiments, each of said at least two universal probe binding motifs comprises a unique nucleotide sequence when said plurality of said non-extendible primers are used.

Disclosed herein, in some aspects, is a kit for use in determining the presence or absence of a target nucleic acid in the presence of a non-target nucleic acid, said kit comprising: (A) a forward primer comprising a first region configured to hybridize to said target nucleic acid under amplification conditions and configured to not hybridize to said non-target nucleic acid under said amplification conditions, and a second region configured to not hybridize to said target nucleic acid under said amplification conditions; (B) a signal generating nucleic acid probe that anneals to said second region, or regions complementary thereto, when subjected to said amplification conditions; and (C) instructions. In some embodiments, said signal generating nucleic acid probe is configured, upon introduction of a target nucleic acid and exposure to appropriate conditions, to be degraded, and a signal is generated if said target nucleic acid is present in said mixture; and the presence or absence of said signal is configured to be detected.

Disclosed herein, in some aspects, is a system for determining the presence or absence of a target nucleic acid in the presence of a non-target nucleic acid, said system comprising: (A) a reaction vessel configured to receive: (i) said sample; (ii) a forward primer comprising a first region configured to hybridize to said target nucleic acid under amplification conditions and configured to not hybridize to said non-target nucleic acid under said amplification conditions, and a second region configured to not hybridize to said target nucleic acid under said amplification conditions; and (iii) a signal generating nucleic acid probe, where said signal generating nucleic acid probe anneals to said second region, or regions complementary thereto, when subjected to said amplification conditions; (B) a thermocycler configured to subject said mixture to said amplification conditions, said amplification conditions appropriate to amplify said target nucleic acid with an amplification reaction, thereby amplifying said target nucleic acid such that said signal generating nucleic acid probe is degraded and a signal is generated if said target nucleic acid is present in said mixture; and (C) a detector configured to detect the presence or absence of said signal, thereby determining the presence or absence of said target nucleic acid in the presence of said non-target nucleic acid.

Disclosed herein, in some aspects, is a system comprising a controller comprising or capable of accessing, computer readable media comprising non-transitory computer-executable instructions which, when executed by at least one electronic processor perform a method comprising: (A) providing a sample comprising, or potentially comprising, said target nucleic acid and said non-target nucleic acid; (B) forming a mixture comprising: (i) said sample; (ii) a forward primer comprising a first region configured to hybridize to said target nucleic acid under amplification conditions and configured to not hybridize to said non-target nucleic acid under said amplification conditions, and a second region configured to not hybridize to said target nucleic acid under said amplification conditions; and (iii) a signal generating nucleic acid probe, where said signal generating nucleic acid probe anneals to said second region, or regions complementary thereto, when subjected to said amplification conditions; (C) subjecting said mixture to said amplification conditions, said amplification conditions appropriate to amplify said target nucleic acid with an amplification reaction, thereby amplifying said target nucleic acid such that said signal generating nucleic acid probe is degraded and a signal is generated if said target nucleic acid is present in said mixture; and (D) detecting the presence or absence of said signal, thereby determining the presence or absence of said target nucleic acid in the presence of said non-target nucleic acid.

Systems and kits as disclosed herein may be configured or constructed such to perform methods as described herein

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 demonstrates how a PCR using allele specific universal tail primers may be used to selectively amplify mutant alleles. FIG. 1a depicts a DNA sample may contains both mutant and wild type alleles. FIG. 1b illustrates how universal tail primers may be specific to and only amplify mutant alleles. FIG. 1c shows the incorporation of an additional primer may be used to cleave TaqMan® probes that are complementary to universal tail primer.

FIG. 2 reveals various strategies can may be used to select primer and probe locations for reporting the presence of the mutant allele. FIG. 2a exhibits how a mutant-specific reporting probe may be designed to be specific to the mutant allele. FIG. 2b establishes a second mutant-specific primer may be used to digest a reporting probe. FIG. 2c discloses a universal primer sharing homology with the universal tail primer may similarly be used to digest a mutant-specific probe.

FIG. 3 discloses a universal tail primer may be designed to have multiple homology regions: (i) specific to an allele; (ii) specific to a mutation reporting tag; and (iii) specific to an additional universal primer.

FIG. 4 illustrates how universal tail primers incorporating three distinct homology regions (one specific to the mutant allele, one engineered to label the mutant allele, and one specific to a universal primer) can be read out in multiple configurations.

FIGS. 5A and 5B shows a universal tailed primer with a universal probe binding motif, where the tailed primer sequence contains the target-specific sequence at the 3′ end, and a universal primer binding motif at the 5′ end, and where the universal probe binding motif is flanked between the target-specific sequence and universal primer binding motif.

FIG. 6 introduces a universal tailed primer comprising at least two universal probe binding motifs. The tailed primer sequence contains the target-specific sequence at the 3′ end, and a universal primer binding motif at the 5′ end, and where at least two universal probe binding motifs are flanked between the target-specific sequence and universal primer binding motif.

FIG. 7 shows a computer control system that is programmed or otherwise configured to implement methods provided herein.

### DETAILED DESCRIPTION

The following description provides specific details for a comprehensive understanding of, and enabling description for, various embodiments of the technology. It is intended that the terminology used be interpreted in its broadest reasonable manner, even where it is being used in conjunction with a detailed description of certain embodiments.

Before describing the present teachings in detail, it is to be understood that the disclosure is not limited to specific compositions or process steps, and as such, may vary. As used herein, the singular forms “a,” “an,” and “the” are intended to include the plural forms as well, unless the context clearly indicates otherwise. Furthermore, to the extent that the terms “including,” “includes,” “having,” “has,” “with,” “such as,” or variants thereof, are used in either the specification and/or the claims, such terms are not limiting and are intended to be inclusive in a manner similar to the term “comprising.” Unless specifically noted, embodiments in the specification that recite “comprising” various components are also contemplated as “consisting of” or “consisting essentially of” the recited components.

Polymerase Chain Reaction (PCR) is a method of exponential amplification of a specific target nucleic acid in a reaction mix using a nucleic acid polymerase and primers. Primers are short single stranded oligonucleotides exhibiting complementary to the 3′ sequences of the positive and negative strand of the target sequence. The reaction mix is cycled in repeated heating and cooling steps. The heating cycle denatures or separates the double stranded target nucleic acid into single stranded templates. In the cooling cycle, the primers bind to complementary sequence on each template strand. After the template is primed, the nucleic acid polymerase creates a copy of the original template. Repeated cycling exponentially amplifies the target 2-fold with each cycle; yielding approximately a billion-fold increase of the target sequence in 30 cycles (Saiki et al. 1988).

Real-Time PCR (qPCR) is a process of monitoring a PCR reaction by recording the fluorescence generated either by an intercalating dye such as SYBR Green or a target-specific reporter probe at each cycle. This is generally performed on a Real-Time PCR instrument that executes thermal cycling of the mixture to complete the PCR cycles and at a specified point in each cycle measures the fluorescence of the mixture in each channel through a series of excitation/emission filter sets.

Multiplex analysis of multiple target nucleic acids in a single measurement may be performed by encoding each target nucleic acid to a unique intensity value or range of values. For example, when detecting multiple target nucleic acids in a sample using a single measurement, oligonucleotide probes may be provided at varying concentrations, such that the intensity of each signal generated from the probes, both individually and in combination, is unique.

The term “channel,” “color channel,” or “optical channel”, as used herein, refers to a range of wavelengths. The range of wavelengths may be set or determined based on particular filters, which remove or “filter out” particular wavelengths. The terms “channel,” “color channel,” and “optical channel” may be used interchangeably.

### Overview

Single nucleotide polymorphisms (SNPs) are a type of polymorphism involving variation of a single nucleotide—adenine (A), guanine (G), thymine (T), or cytosine (C)—in a segment of a DNA molecule. SNPs may be a switch (i.e., substitution), removal (i.e., deletions) or addition (i.e., insertion) of a single nucleotide base within a polynucleotide sequence. SNPs may fall within coding or non-coding sequences of genes. Naturally, genotypic alterations may consequently influence one's physiological responses to drugs and/or increase the likelihood of disease. Accordingly, identifying SNPs that have known corollary relationships and/or associations with disease states are beneficial for diagnostic purposes.

Certain SNPs may be more prevalent in certain populations. Within a population and/or geographical area, SNPs can be assigned a minor allele frequency, i.e., the frequency a SNP occurs a particular locus. As SNPs common in one geographical area or ethnic group may not be prevalent in other geographical regions or ethnic groups, comparative statistics using SNPs may provide additional diagnostic benefits.

Methods to distinguish and/or detect SNPs need to be highly specific and sensitive. Polymerase chain reaction (PCR) provides the necessary analytical performance for many molecular analyses. Real-Time PCR (qPCR) is a process of monitoring a PCR reaction by recording the fluorescence generated either by an intercalating dye or a target-specific reporter probe at each cycle. In a singleplex PCR, a single target is amplified. In a multiplex PCR, multiple targets are amplified. In fluorescently multiplexed PCR, detection of multiple target nucleic acid sequences is accomplished by associating each target nucleic acid with a distinct fluorescent tag such as a TaqMan® probe, FRET probe, or a molecular beacon.

Conventional PCR-based methods for SNP detection broadly fall into two methodological categories: (1) Allele-specific PCR using primers matched with a substituted nucleotide to block the nontargeted template; or (2) melting curve analysis combined with the real-time PCR. However, the aforementioned PCR methods for SNP detection each suffer from limitations that inhibit broad adoption. For example, allele-specific PCR utilizes nucleic acid or protein blockers to selectively inhibit the amplification of the wild type allele. These blockers bind to the priming location with stronger kinetics than the primers themselves. Importantly, the design of blockers is not straightforward, and often precludes their use in a number of applications. Additionally, while primers with terminal fluorescent base pairs can be used to identify particular point mutations, this method requires the resulting fluorescently labeled, non-extendible product be separated using an electrophoretic column

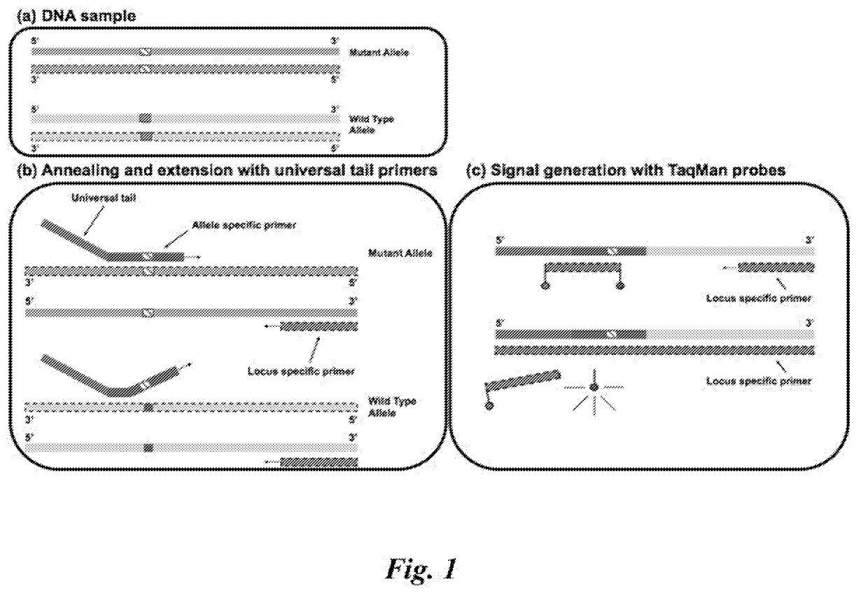

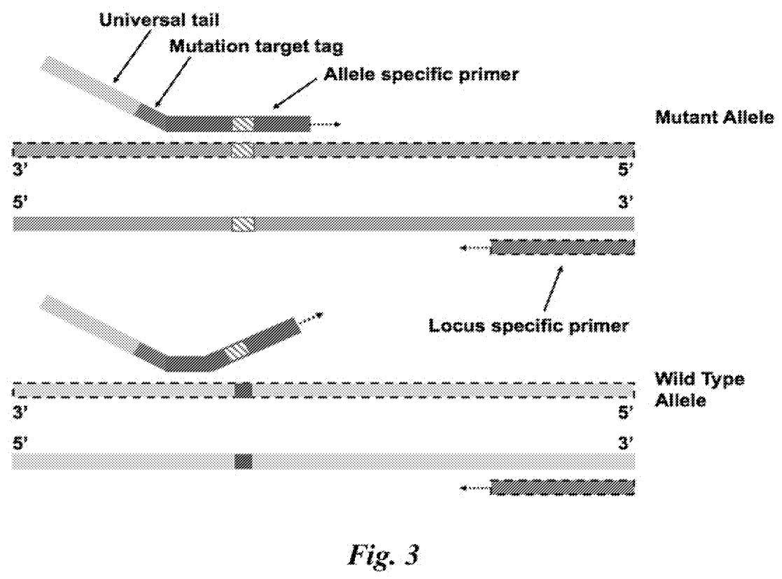

The present disclosure introduces a novel means for determining the presence of a target nucleic acid in the presence of a non-target nucleic acid using a target-specific primer that can discriminate between a target and a non-target without the use of blocking agents. As used herein the term “target” and “target nucleic acid” can be used interchangeably. As used herein, the term “non-target” and “non-target nucleic acid” can be used interchangeably. Specifically, target-specific primers—specific to and only able to amplify target alleles—may be used to discriminate the fluorescence signal of target amplicons (see FIGS. 1 and 3).

Specifically, the disclosure may be used for discriminating between a mutant and wild type allele which differ by as few as a single base pair, for example, a SNP. The target nucleic acid and the non-target nucleic acid may differ by only one base pair or nucleotide. The target nucleic acid and the non-target nucleic acid may differ by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 90, 100, or less base pairs. The target nucleic acid and the non-target nucleic acid may differ by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 90, 100, or more base pairs

The target-specific primers of the present disclosure may be constructed as to comprise two regions: a first region with complementarity or homology to the target configured to hybridize to the target and a second which is not complementary or homologous to the target or not configured to hybridize to the target. The second region may comprise a sequence that may be shared with other primers. The second region may be constructed out of any sequence, as described elsewhere herein, that is otherwise not complementary or homologous to the target nucleic acid and is not configured to hybridize or bind to the target. This sequence and corresponding region may be referred to as universal in that it is a sequence that is agnostic or independent of the target and can be appended to any target specific primer sequence, thereby being “universal”, and is on the end of a primer sequence (thereby being a “tail) that comprises a region specific to a target nucleic acid. A primer that comprise this universal tail may be referred to as a universal tail primer.

In some embodiments, a universal tail primer may comprise: a target-specific segment with complementary to the target nucleic acid; a target-specific tail segment not complementary to the target nucleic acid; and a universal tail segment not complementary to the target. The target-specific segment with complementary to the allele target may be located on the 3′ end of the primer, whereas the universal tail segment may be located on the 5′ end of the primer. The target-specific tail segment may be flanked between the 3′ target-specific segment and 5′ universal tail segment. The universal tail primer can be designed to have multiple homology regions: one specific to an allele, one specific to a mutation reporting tag, and one specific to an additional universal primer.

Universal tails attached to target-specific primer sequences provide several benefits. First, universal tails disconnect the signal generation from the target sequence, thereby reducing required amplicon length and homology between inclusivity targets. Secondly, universal tails allow for multiple targets to have the same universal tail, thus generating the same signal from non-homologous sequences (ideal for divergent sequences). Third, universal tails increase specificity for target sequence as significantly lower concentration of target-specific primer in the tailed primer is required. Finally, universal tails shorten assay design time since the universal primer and probes are assay independent.

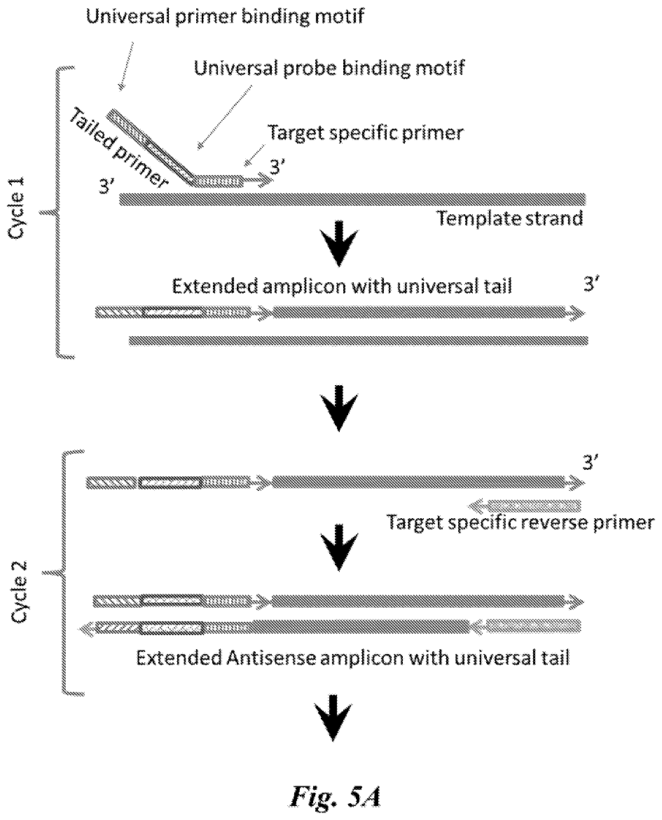

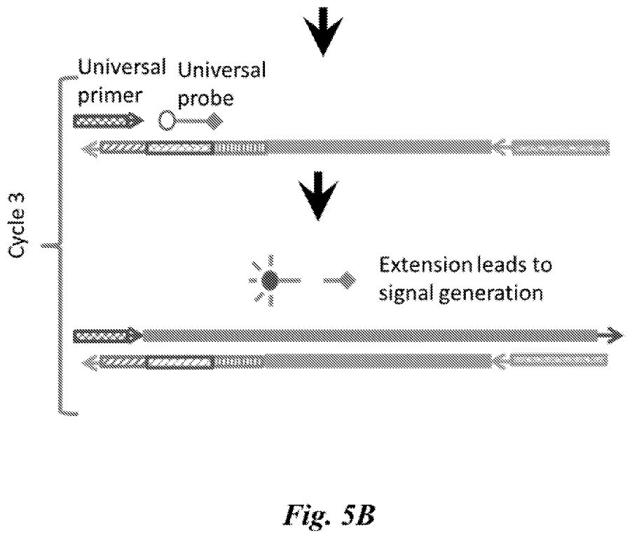

In some embodiments, the universal tailed primers may further incorporate multiple probe binding motifs. In a simplified example of a universal tailed primer, a target-specific “forward” primer with a synthetic sequence tail is paired with a target-specific antisense “reverse” primer to allow for exponential amplification. A universal probe binds to the antisense of the tail and universal forward primer which can bind to the antisense of the tailed primer. The tailed primer sequence contains the target-specific sequence at the 3′ end, a probe binding motif which is in the same sense as the signal generating TaqMan Probe, and a universal primer binding motif at the 5′ end which is in the same sense as a universal primer (see FIG. 5). When the tail is incorporated into the PCR amplicon, the reverse primer generates an antisense strand which can bind the universal primer and probe. Thus, the universal primer becomes the dominant driver of the amplification of the target sequence instead of the tailed primer and hydrolyses the universal probe.

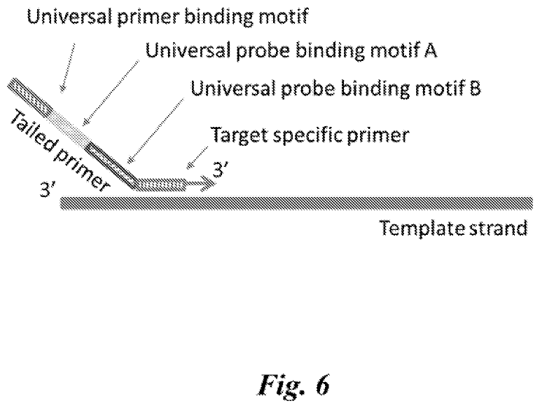

Including at least two probe binding motifs in the universal tailed primer provides additional, novel benefits (see FIG. 6). The inclusion of additional binding motifs allows for extensive barcoding of individual or groups of targets for HDPCR or Resilient coding schemes, thereby enhancing universal reporter systems. In some embodiments, the probe binding motifs may be different. The inclusion of multiple probe binding motif allows a target to be detected across multiple color channels at the same or different levels without competition between the probes. Multiple probe signals will be in sync across detection channels since they are generated of the template allowing for curve matching algorithms to increase call confidence, whereas single targets can be detected at a non-primary level within the same channel if it included multiple different probes with the same dye. In some embodiments, the probe binding motifs may be repeats. Repeat of the same probe binding motif allow for integer fold increases in the fluorescence added with each cycle per amplicon; thereby reducing the cycle threshold crossing point 1 cycle for every two-fold increase in the probe binding motif without changing the endpoint intensity level.

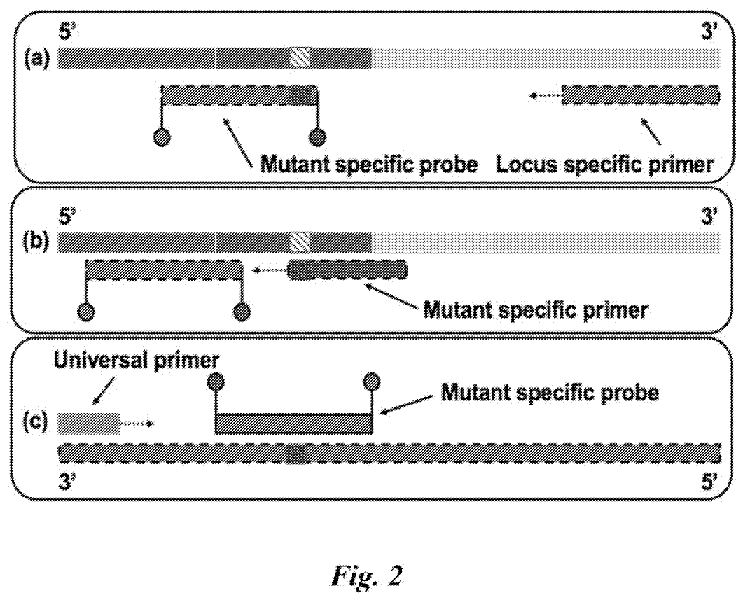

In some embodiments, a reporting probe can be designed to be specific to a target. (see FIG. 2). In some embodiments, the signal generating nucleic acid probe is a target-specific probe. In some embodiments, the target-specific probe has the same sense as the target specific primer and contains segments of the 3′ end of the universal tail, the entire target-specific tail, and a portion complementary to the target-specific segment. In other embodiments, target-specific probe contains segments of the 3′ end of the universal tail, and a portion of the target-specific tail. In some embodiments, the probe binds to the product of an amplification reaction generated by second strand synthesis from a primer common to the target locus. In other embodiments, the signal generating nucleic acid probe is a universal probe. A universal probe may bind a universal tail. A universal probe may bind a universal tail regardless of the sequence of the target.

Digesting a signal generating probe in a single reaction (without the use of blockers) provides for greater specificity than conventional allele-specific PCR methods to detect SNPs employing primers matched with substituted nucleotides to block the nontargeted template. In some embodiments, a second target-specific primer can be used to digest a reporting probe. In other embodiments, a universal primer sharing homology with the universal tail primer may be used to digest a target-specific probe. In some embodiments, the universal primer has the same sequence of the whole or a 5′ portion of the universal tail and is present in the reaction for TaqMan® based qPCR probes.

Notably, universal tail methodology may be used in conjunction with other target specific methods to increase the competitive amplification of the target over the non-target. For example, in one embodiment, a RNA, DNA or unnatural base can be substituted into an unextendible target specific, universal-tailed primer. The non-extendible nucleic acid primer may comprises a target-specific RNA base, DNA base, or unnatural base, configured to, when said first region is hybridized to said target nucleic acid, form a base hybrid pair at a target base of said target nucleic acid and not form said base hybrid pair at a corresponding base in the non-target nucleic acid. The base hybrid pair may be a RNA:DNA pair, an DNA: unnatural base pair, or a RNA:unnatural base pair. For example, in one embodiment, an RNA, DNA or unnatural base can be substituted into an unextendible target specific, universal-tailed primer at the location complementary to a mutation or SNP. When the universal tail primer binds to the mutant allele (due to base-pair homology), it would form an RNA:DNA hybridization at the location of the mutation or a divergence of sequence between a target and non-target. This bond can then be cleaved by an additional enzyme, ribonuclease H. After cleavage, the universal tail primer can then be extended by DNA polymerase. In other embodiment, the target nucleic acid may be an RNA, for example an mRNA. The primer may comprise a DNA such to generate a RNA:DNA base hybrid pair.

The use of universal tail primers further provides for the detection of multiple mutations in a single-color channel. For example, a single universal tail may be used in combination with multiple SNP-specific tails, or the multiple universal tails may be used in combination with multiple SNP-specific tails.

FIG. 1 shows the detection of a DNA sample containing a mutant allele (target nucleic acid) and a wild type (non-target nucleic acid). FIG. 1B shows a primer comprising a universal tail and an allele specific primer (target specific primer) which anneals to the mutant allele (target) and not the wild type (non target). Upon extension or amplification using the primer, a nucleic acid strand with the universal tail is formed. The strand may then be detected using a variety of methods. FIG. 1C shows the use of probe configured to bind to the new strand, wherein the probe is complementary to a portion of the universal tail as well as to a portion the target specific primer sequence. Specifically, the probe may include a sequence that is homologous or complementary to an SNP or mutation in the target nucleic acid. Using a locus specific primer (which could bind both the wild type and mutant), an extension reaction is conducted, and the signal can be generated by the digestion of the probe. The wild type (non target) does not produce any signal as it has not been amplified, and the probe is specific to the amplified target comprising the universal tail and mutation.

FIG. 2 shows additional embodiments for signal generation after extension of the target or mutant nucleic acid. FIG. 2A shows a similar embodiment described in FIG. 1C. FIG. 2B shows an embodiment in which a mutant specific primer (i.e. one that contains the SNP or mutation) is used along with a probe that is configured to bind a portion of the universal tail as well as to a portion the target specific primer sequence. FIG. 2C shows the use of a universal primer which can to hybridize to a portion of the universal tail and the probe is a mutant specific probe which is homologous or complementary to a region of the target nucleic acid.

FIG. 3 shows a similar embodiment to FIG. 1B with an additional mutation target tag (target specific tail segment). This embodiment may operate similarly as depicted in FIG. 1B-C. The addition of the mutation tag or target specific tail segment may act as an additional identifier for observing the target nucleic acid.

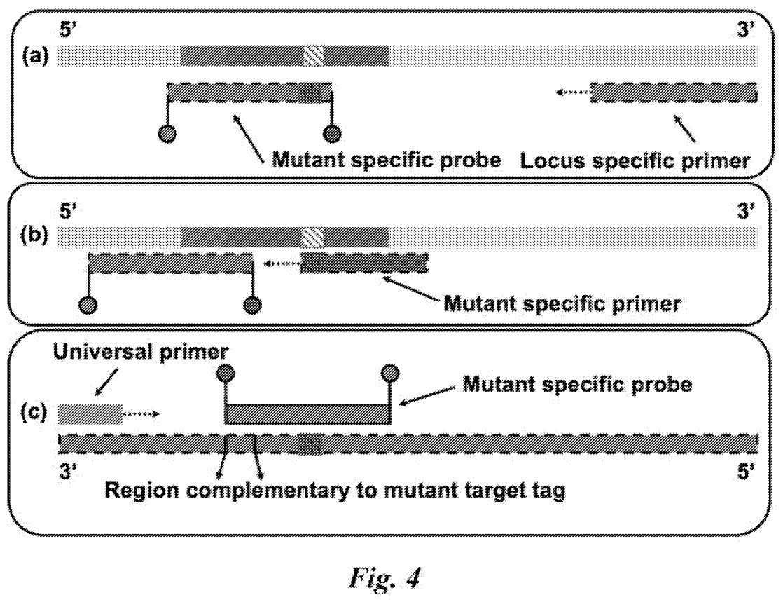

FIG. 4A-C shows similar embodiments and FIG. 2A-C, except with the addition of a mutation target tag (target-specific tail segment), and operate similarly as described for FIG. 2A-C

FIG. 5A-B show the use of a primer with a universal primer binding motif and a universal probe binding motif. Cycle 1 shows the extension of the primer to generate a amplicon with a universal tail. Cycle 2 uses the extended amplicon with a universal tail and generates an antisense amplicon using a target-specific reverse primer. FIG. 5B shows the third cycle of the reaction in which a signal may be generated using the antisense amplicon of FIG. 5A. A universal probe that binds or hybridizes to the universal probe binding motif, and a universal primer that binds or hybridizes to a universal primer motif are used. The signal from the probe is then generated by the extension reaction.

FIG. 6 shows and embodiment in which a primer has two universal probe binding motifs (universal probe binding motif A and universal probe binding motifs B). Two of the same or different probes may bind to theses motifs allow the signal to be displayed in multiple channel. The primer shown in FIG. 6 may be used in as shown in FIG. 5A-B.

### Assays

In some aspects, the present disclosure provides assays for unambiguously detecting the presence or absence of multiple target nucleic acids in a mixture. Target nucleic acid detection may be accomplished by the use of two or more reactions. For example, an assay for measuring a plurality of target nucleic acids may comprise a first reaction and a second reaction. The results of the first and second reactions may together unambiguously detect the presence or absence of each of the target nucleic acids.

Any number of target nucleic acids may be detected using assays of the present disclosure. In some cases, an assay may unambiguously detect at least two, three, four, five, six, seven, eight, nine, ten, 11, 12, 13, 14, 15, 20, 30, 40, 50 target nucleic acids, or more. In some cases, an assay may unambiguously detect at most 50, 40, 30, 20, 15, 14, 13, 12, 11, ten, nine, eight, seven, six, five, four, three, or two target nucleic acids. An assay may comprise any number of reactions, where the results of the reactions together identify a plurality of target nucleic acids, in any combination of presence or absence. An assay may comprise two, three, four, five, six, seven, eight, nine, ten reactions, or more.

Reactions may be performed in the same mixture solution volume. For example, a first reaction may generate a fluorescent signal in a first color channel, while a second reaction may generate a fluorescent signal in a second color channel, thereby generating two measurements for comparison. Alternatively, reactions may be performed in different mixture solution volumes. For example, a first reaction may be performed in a first mixture solution volume and generate a fluorescent signal in a given color channel, and a second reaction may be performed in a second mixture solution volume and generate a fluorescent signal in the same color channel or a different color channel, thereby generating two measurements for comparison.

Assay reactions as described herein may be conducted in parallel. In general, parallel assay reactions are reactions that occur in the same reaction vessel and at the same time. For example, parallel nucleic acid amplification reactions may be conducted by including reagents used for each nucleic acid amplification reaction in a reaction vessel to obtain a reaction mixture and subjecting the reaction mixture to conditions used for each nucleic acid amplification reaction. Any suitable number of nucleic acid amplification reactions may be conducted in parallel. In some cases, at least one, two, three, four, five, six, seven, eight, nine, ten, 11, 12, 13, 14, 15, 16, 17, 18, 19, 20, or more nucleic acid amplification reactions are conducted in parallel.

In some aspects, the disclosed method employs the use of oligonucleotide probes which may be labeled with a fluorophore. Fluorescent molecules may be excited at a wavelength at emit light at another wavelength. The fluorescent molecules may be visible to the naked human eye. The fluorescent molecules may visible or identified via spectroscopic methods such to analyze the wavelength of light that are transmitted or absorbed by a solution comprising a fluorescent molecule. The fluorophores may be capable of being detected in a single optical channel (e.g., the fluorophores may each comprise similar emission wavelength spectra, such that they can be detected in a single optical channel). The fluorophores may be capable of being detected in a more than one optical channel (e.g., the fluorophores may each comprise emission wavelength spectra, such that they can be detected across one or more optical channels). The fluorescent molecules may have a distinct or known signature of excitation or emission wavelength of electromagnetic radiation. The detection of a fluorescent molecule signature may comprise identifying an amplitude or amplitudes of signal at different wavelengths. The fluorescent molecule signature may comprise a signal at wavelengths that do not overlap with wavelengths that may be generated by reagents in the chemical composition. In some cases, the excitation wavelength of the molecule may comprise a signal that does not overlap with wavelengths that may be generated by reagents in the chemical composition. In some cases, the signals of the reaction and the fluorescent molecule may be simultaneously detected. In some cases, N signals may be generated from the oligonucleotide probes. Each signal of the N signals may correspond to the presence of a unique combination of target nucleic acids.

A reaction may comprise generating a cumulative signal measurement. Assays of the present disclosure may comprise comparing two or more cumulative signal measurements to unambiguously detect any combination of target nucleic acids in a mixture. A cumulative signal measurement may comprise one or more signals generated from one or more probes provided to a mixture solution. A cumulative signal measurement may be a signal intensity level which corresponds to the sum of signals generated from multiple oligonucleotide probes.

### Amplification

In some aspects, the disclosed methods comprise nucleic acid amplification. Amplification conditions may comprise thermal cycling conditions, including temperature and length in time of each thermal cycle. An amplification reaction may be a PCR reaction. An amplification reaction may be an RT-PCR reaction. An amplification reaction may be a digital PCR reaction. The use of particular amplification conditions may serve to modify the signal intensity of each signal, thereby enabling each signal to correspond to a unique combination of target nucleic acids. Amplification may comprise generating amplicons with an additional sequence, for example the addition of a universal tail segment or a target specific tail segment. Amplification may comprise using enzymes such to produce additional copies of a nucleic. The amplification reaction may comprise using primers as described elsewhere herein. The primers may use specific sequences to amplify a specific sequence. The primers may amplify a specific sequence by hybridizing to a sequence upstream and downstream of the primers and result in amplifying the sequence inclusively between the upstream and downstream primer. The amplification reaction may comprise the use of nucleotide tri-phosphate reagents. The nucleotide tri-phosphate reagents may comprise using deoxyribo-nucleotide tri-phosphate (dNTPs). The nucleotide tri-phosphate reagents may be used as precursors to the amplified nucleic acids. The amplification reaction may comprise using oligonucleotide probes as described elsewhere herein. The amplification reaction may comprise using enzymes. Non-limiting examples of enzymes include thermostable enzymes, DNA polymerases, RNA polymerases, and reverse transcriptases. The amplification reaction may comprise generating nucleic acid molecules of a different nucleotide types. For example, a target nucleic acid may comprise DNA and an RNA molecule may be generated. In another example, an RNA molecule may be subjected to an amplification reaction and a cDNA molecule may be generated.

### Thermal Cycling

Methods of the present disclosure may comprise thermal cycling. Thermal cycling may comprise one or more thermal cycles. Thermally cycling may be performed under reaction conditions appropriate to amplify a template nucleic acid with PCR. Amplification of a template nucleic acid may require binding or annealing of primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a target nucleic acid. Appropriate buffer conditions may, in some cases, be such that the appropriate salts are present in a buffer used during thermal cycling. Appropriate salts may include magnesium salts, potassium salts, ammonium salts. Appropriate buffer conditions may be such that the appropriate salts are present in appropriate concentrations. Appropriate reagents for amplification of each member of a plurality of target nucleic acids with PCR may include deoxytriphosphates (dNTPs). dNTPs may comprise natural or non-natural dNTPs including, for example, dATP, dCTP, dGTP, dTTP, dUTP, and variants thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (Ct) of less than 100, 75, 70, 65, 60, 55, 50, 45, 40, 35, 30, 25, 20, 15, 10, or 5.

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less

### Target Nucleic Acids

In some cases, a target nucleic acid may comprise deoxyribonucleic acid (DNA). DNA may be any kind of DNA, including genomic DNA. A target nucleic acid may be viral DNA. A target nucleic acid may comprise ribonucleic acid (RNA). RNA may be any kind of RNA, including messenger RNA, transfer RNA, ribosomal RNA, and microRNA. RNA may be viral RNA. A target nucleic acid may comprise a gene whose detection may be useful in diagnosing one or more diseases. A gene may be a viral gene or bacterial gene whose detection may be useful in identifying the presence or absence of a pathogen in a subject.

Target nucleic acids may be of any length. A target nucleic acid may be, for example, up to 1, 2, 3, 4, 5, 10, 20, 50, 100, 500, 1000, 5000, 10000, 50000, or 100000 nucleotides, or more. In some instances, a target nucleic acid may be a gene. A target nucleic acid may be an allele. A target nucleic acid may be a mutant of a wild type allele. A target nucleic acid may be a mutant and a non-target nucleic acid may be the corresponding wild type sequence. A target nucleic acid may comprise a SNP. A target nucleic acid may comprise a gene whose detection may be useful in diagnosing one or more diseases. A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses) in a subject.

Target nucleic acids may be of various concentrations in the reaction. The nucleic acid sample may be diluted or concentrated to achieve different concentrations of nucleic acids. The concentration of the nucleic acids in the nucleic acid sample may at least 0.1 nanograms per microliter (ng/μL), 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or more. In some cases, the concentration of the nucleic acids in the nucleic acid sample may be at most ng/μL, 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or less.

### Sample

A target nucleic acid of the present disclosure may be derived from a biological sample. A biological sample may be a sample derived from a subject. A biological sample may comprise any number of macromolecules, for example, cellular macromolecules. A biological sample may be derived from another sample. A biological sample may be a tissue sample, such as a biopsy, core biopsy, needle aspirate, or fine needle aspirate. A biological sample may be a fluid sample, such as a blood sample, urine sample, or saliva sample. A biological sample may be a skin sample. A biological sample may be a cheek swab. A biological sample may be a plasma or serum sample. A biological sample may comprise one or more cells. A biological sample may be a cell-free sample. A cell-free sample may comprise extracellular polynucleotides. Extracellular polynucleotides may be isolated from a bodily sample that may be selected from the group consisting of blood, plasma, serum, urine, saliva, mucosal excretions, sputum, stool and tears.

### Sample Processing

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest.

### Nucleic Acid Enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V.

A nucleic acid enzyme may be a polymerase (e.g., a DNA polymerase). A polymerase may be Taq polymerase or a variant thereof. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases. A nucleic acid enzyme may be capable, under appropriate conditions, of degrading an oligonucleotide probe. For example, a nucleic acid enzyme may be a polymerase and comprise exo activity and degrade a probe resulting in a detectable signal. A nucleic acid enzyme may be capable, under appropriate conditions, of releasing a quencher from an oligonucleotide probe.

### Reactions

A reaction may comprise contacting target nucleic acids with one or more oligonucleotide probes. A reaction may comprise the use of intercalating dyes, thereby removing the need for probes. A reaction may comprise contacting a mixture solution volume (e.g., a droplet, well, tube, etc.) with a plurality of oligonucleotide probes, each corresponding to one of a plurality of target nucleic acids, to generate a plurality of signals generated from the plurality of oligonucleotide probes. A reaction may comprise an amplification reaction. A reaction may comprise an extension reaction. A reaction may comprise polymerase chain reaction (PCR). A reaction may comprise reverse-transcriptase-polymerase chain reaction (PCR). A reaction may comprise a quantitative PCR reaction (qPCR).

Reactions may be performed by subjecting a mixture to conditions sufficient for the reaction to occur. For example, an amplification reaction may occur by subjecting a mixture to amplification conditions comprising thermal cycling. The amplification conditions may comprise that each thermal cycle is performed at an annealing temperature appropriate for annealing said forward primer to said target nucleic acid. The amplification conditions may comprise the addition of an enzyme, a salt, a buffer, dNTPs, or other substrates. The conditions may comprise a pH. The conditions may comprise a temperature. The pH may be suitable to allow a reaction to proceed. The reaction conditions may comprise changing a pH such that the reaction may proceed or is otherwise initiated. The reaction conditions may comprise changing a temperature such that the reaction may proceed or is otherwise initiated.

### Primers

In various aspects disclosed elsewhere herein, primers are used. A primer may also be referred to herein as an “amplification oligomer”, “oligonucleotide primer”, or “nucleic acid primer”. The primer may be a deoxyribonucleic acid. A primer may be a ribonucleic acid. A primer may comprise one or more non-natural nucleotides. A non-natural nucleotide may be, for example, deoxyinosine.

A primer may be a forward primer. A primer may be a reverse primer. A primer may be between about 5 and about 50 nucleotides in length. A primer may be at least 5, 10, 11, 12, 13, 14, 15, 16, 17, 18, 19, 20, 25, 30, 35, 40, 45, or 50 base pairs in length, or more. A primer may be at most 50, 45, 40, 35, 30, 25, 20, 19, 18, 17, 16, 15, 14, 13, 12, 11, 10, or 5 nucleotides in length. A primer may be about 5, 10, 11, 12, 13, 14, 15, 16, 17, 18, 19, 20, 25, 30, 35, 40, 45, or 50 base pairs in length.

A pair of primers may comprise a forward primer and a reverse primer. A forward primer may be configured to hybridize to a first area (e.g., a 3′ end) of a nucleic acid sequence, and a reverse primer may be configured to hybridize to a second area (e.g., a 5′ end) of the nucleic acid sequence, thereby being configured to amplify the nucleic acid sequence under conditions sufficient for nucleic acid amplification. Different pairs of primers may be configured to amplify different target nucleic acid sequences.

A mixture may comprise a plurality of forward primers. A plurality of forward primers may be a deoxyribonucleic acid. Alternatively, a plurality of forward primers may be a ribonucleic acid. A plurality of forward primers may be between about 5 and about 50 nucleotides in length. A plurality of forward primer may be at least 5, 10, 11, 12, 13, 14, 15, 16, 17, 18, 19, 20, 25, 30, 35, 40, 45, or 50 base pairs in length, or more. A plurality of forward primer may be at most 50, 45, 40, 35, 30, 25, 20, 19, 18, 17, 16, 15, 14, 13, 12, 11, 10, or 5 nucleotides in length.

A mixture may comprise a plurality of reverse primers. A plurality of reverse primers may be a deoxyribonucleic acid. Alternatively, a plurality of reverse primers may be a ribonucleic acid. A plurality of reverse primers may be between about 5 and about 50 nucleotides in length. A plurality of reverse primer may be at least 5, 10, 11, 12, 13, 14, 15, 16, 17, 18, 19, 20, 25, 30, 35, 40, 45, or 50 base pairs in length, or more. A plurality of reverse primer may be at most 50, 45, 40, 35, 30, 25, 20, 19, 18, 17, 16, 15, 14, 13, 12, 11, 10, or 5 nucleotides in length.

In some aspects, a mixture may be subjected to conditions sufficient to anneal a primer to a nucleic acid molecule. In some aspects, a mixture may be subjected to conditions sufficient to anneal a plurality of primers to a nucleic acid molecule. In some aspects, a mixture may be subjected to conditions sufficient to anneal a plurality of primers to a plurality of target nucleic acids. The mixture may be subjected to conditions which are sufficient to denature nucleic acid molecules. Subjecting a mixture to conditions sufficient to anneal a primer to a target nucleic acid may comprise thermally cycling the mixture under reaction conditions appropriate to amplify the target nucleic acid(s) with, for example, polymerase chain reaction (PCR).

Conditions may be such that a primer pair (e.g., forward primer and reverse primer) are degraded by a nucleic acid enzyme. A primer pair may be degraded by the exonuclease activity of a nucleic acid enzyme. A primer pair may be degraded by the RNase activity of a nucleic acid enzyme. Degradation of the primer pair may result in release of the primer. Once released, the primer pair may bind or anneal to a template nucleic acid.

Primers may be complementary or, homologous to, bind, or hybridize to a variety of sequences. For example, a primer be complementary or, homologous to, or bind, or hybridize to a particular locus and allow amplification of a locus. A primer be complementary or, homologous to, or bind, or hybridize to a specific sequence comprising a mutation or SNP. A primer be complementary or, homologous to, or bind or hybridize to a universal tail sequence. A primer be complementary or, homologous to, or bind, or hybridize to target-specific tail segment sequence. A primer may be complementary or, homologous to, or be able to bind, or hybridize to both a target and a non-target sequence. For example, a primer may bind a region that is the same between and target and non-target thereby allowing a non-target and target to be amplified. Although a primer may be able to hybridize to a sequence of both a target and non-target, the presence, lack thereof, of a corresponding forward or reverse may allow or prevent the amplification of the target or non-target.

The primer may comprise a tail sequence or region. The tail sequence or region may be a universal tail. A primer may comprise a target specific tail segment. A target specific tail sequence may be referred to elsewhere herein as a “mutation target tag” as described in FIGS. 3 and 4. For example, the target-specific tail segment may be not complementary or homologous to the target nucleic acid but when amplified may append an additional sequence that corresponds to the target. The target specific tail segment may be used to identify the target nucleic acid without probes to the target nucleic acid and instead may be identified via a probe or recognition of the sequence complementary or homologous to the target-specific tail segment. The primer may comprise a universal primer binding motif. This universal primer binding motif may be a universal tail. The universal primer binding motif may be used to amplify the target nucleic acid after an amplicon has been generated comprising the universal binding motif. A universal primer may bind to the universal primer binding motif and allow the amplification of any nucleic acid with the universal primer binding motif. For example, multiple target nucleic acids of different sequence may be amplified using primers comprising the universal primer binding motif A universal primer may then be used to amplify all the generated amplicons derived from the target nucleic acids regardless of the original sequences of the target nucleic acids. The primer may comprise a universal probe binding motif. This universal probe binding motif may be a universal tail. The universal probe binding motif may be used to detect a target nucleic acid after an amplicon has been generated comprising the universal probe binding motif. A universal probe may bind to the universal primer binding motif and allow the detection of any nucleic acid with the universal probe binding motif. For example, multiple target nucleic acids of different sequence may be amplified using primer comprising the universal probe motif, thereby appending the universal probe binding motif to amplicon derived from the target nucleic acids. The targets may then be detected using probes that are configured to bind the universal probe binding motif A universal probe may then be used to detect all the generated amplicons derived from the target nucleic acids regardless of the original sequences of the target nucleic acids. Primers may comprise more than one universal probe binding motif. For example, a primer may comprise 2 universal probe binding motifs. A primer may comprise more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100, or more, universal probe binding motifs. The universal probe binding motifs may comprise a same sequence or different sequence. For example, a primer may comprise 2 universal probe binding motifs which both bind the same universal probe. For example, a primer may comprise 2 universal probe binding motifs which bind different universal probes. The different universal probes may generate different signals. The use of multiple universal binding probe binding motifs may allow the samples nucleic acids to be coded or categorized based on the universal probe binding motifs. The use of multiple universal binding probe binding motifs may allow the samples nucleic acids to produce a signal in multiple color channels or at multiple wavelengths.

Primer may comprise various sequences in a particular or specific arrangement. For example, the universal tail segment may be at the 5′ end of a primer. The target specific sequence may be at the 3′ end of the primer. The target-specific tail segment sequence may be between a universal tail segment on the 5′ end and a target specific sequence which is configured to bind the target on the 3′ end. The primer may comprise a universal primer binding motif at the 5′ end. The primer may comprise a universal probe binding motif between a universal probe binding motif on the 5′ end and a target specific sequence on the 3′ end.

In some cases, the primer may be a non-extendible primer. The non extendible primer may comprise a non-extendible base at the 3′ end that is unable to polymerize with another nucleotide, thereby preventing it from being extended. For example, the base may lack a 3′ hydroxyl and have a 3′ hydrogen instead. The non-extendible primer may be subjected to conditions to convert it into an extendible primer. The conversion of a non-extensible primer may be performed by removing the non-extendible base. The non-extendible base may be removed by cleaving the primer at the site of the non-extendible base or 5′ of the non-extendible base such that the new 3′ end of the primer is no longer a non-extendible base. The non-extendible nucleic acid primer may comprises a target-specific RNA base, DNA base, or unnatural base, configured to, when said first region is hybridized to said target nucleic acid, form a base hybrid pair at a target base of said target nucleic acid and not form said base hybrid pair at a corresponding base in the non-target nucleic acid. The base hybrid pair may be a RNA:DNA pair, a DNA:unnatural base pair, or a RNA:unnatural base pair. For example, in one embodiment, a RNA, DNA or unnatural base can be substituted into an unextendible target specific, universal-tailed primer at the location complementary to a mutation or SNP. When the universal tail primer binds to the mutant allele (due to base-pair homology), it would form an RNA:DNA hybridization at the location of the mutation or a divergence of sequence between a target and non-target. This bond can then be cleaved by subjecting the primers to a cleavage condition. The cleavage condition may be the addition of an additional enzyme. For example, the additional enzyme may be a ribonuclease H and recognize an RNA:DNA duplex or base hybrid pair. After cleavage, the universal tail primer can then be extended by DNA polymerase. In other embodiment, the target nucleic acid may be an RNA, for example an mRNA. The primer may comprise a DNA such to generate an RNA:DNA base hybrid pair.

The primers may be configured such to anneal and allow an extension or amplification reaction to proceed and digest nucleic acid probe. For example, the primer may be configured to anneal or hybridize to a sequence that is 5′ to another sequence which has been hybridized to a probe. The primer may allow the recruitment of an enzyme which may digest the probe.

In various aspects, multiple primers or a plurality of primers may be used. The plurality of primers may have the same sequences or regions that comprise the same sequence. The plurality of primers may have different sequences or regions that comprise a different sequence. The plurality of primer may have regions that have an identical sequence and other regions that have different sequences. For example, a plurality of primers may comprise primers wherein each second region is identical or dissimilar to another primer. For example, a plurality of primers may comprise primers wherein each universal tail segment is identical or dissimilar to another primer. For example, a plurality of primers may comprise primers wherein each target-specific segment is identical or dissimilar to another primer. For example, a plurality of primers may comprise primers wherein each universal primer binding motif is identical or dissimilar to another primer. For example, a plurality of primers may comprise primers wherein each universal probe binding motif is identical or dissimilar to another primer. For example, a plurality of primers may comprise primers wherein each second region is identical or dissimilar to another primer. A plurality of primers may have a sequence in which is unique to each primer. For example, a region that is configured to be not complementary to the target may all be unique sequence of each of the primers in a plurality of primers, and act an as alternate identifier for observing, monitoring or detecting the target. One of such regions may be the target-specific tail segment.

### Oligonucleotide Probes

In various aspects disclosed elsewhere herein, oligonucleotide probes are used. Mixtures, kits, and compositions of the present disclosure may comprise a target-specific oligonucleotide probe, also referred to herein as a “detection probe” or “probe.” An oligonucleotide probe may be a nucleic acid (e.g., DNA, RNA, etc.). An oligonucleotide probe may comprise a region complementary to a region of a target nucleic acid. The concentration of an oligonucleotide probe may be such that it is in excess relative to other components in a mixture. Oligonucleotide probes may be a signal generating nucleic acid probe. Signal generating nucleic acid probes may comprise features of oligonucleotide probes as described herein, along with the feature of generating a signal upon the application of a stimulus, modification of the probe, or the hydrolysis of the probe. For example, the probe may be hydrolyzed by the exonuclease activity of a polymerase, thereby generating a signal. The signal generating nucleic acid probes may generate any of the signals as described elsewhere herein. For example, said signal generating probes may generate a fluorescent signal. Signal generating probes may comprise a signal tag. The signal tag may generate a signal. The signal tag may generate a signal in response to a reaction or stimulus. For example, a signal tag may generate a signal upon degradation via exonuclease activity.

In some aspects, a mixture may comprise more than one oligonucleotide probe. Multiple oligonucleotide probes may be the same or may be different. An oligonucleotide probe may be at least 5, at least 10, at least 15, at least 20, or at least 30 nucleotides in length, or more. An oligonucleotide probe may be at most 30, at most 20, at most 15, at most 10 or at most 5 nucleotides in length. In some examples, a mixture comprises a first oligonucleotide probe and one or more additional oligonucleotide probes. An oligonucleotide probe may be at least two, three, four, five, six, seven, eight, nine, ten, 20, 30, 40, or 50 nucleotides in length, or more. An oligonucleotide probe may be at most 50, 40, 30, 20, ten, nine, eight, seven, six, five, four, three, or two nucleotides in length.

In some cases, two, three, four, five, six, seven, eight, nine, ten, 11, 12, 13, 14, 15, 20, or more different oligonucleotide probes may be used. Each oligonucleotide probe may correspond to (e.g., capable of binding to) a given region of a target nucleic acid (e.g., a chromosome) in a sample. In one example, a first oligonucleotide probe is specific for a first region of a first target nucleic acid, a second oligonucleotide probe is specific for a second region of the first target nucleic acid, and a third oligonucleotide probe is specific for a third region of the first target nucleic acid. Each oligonucleotide probe may comprise a signal tag with about equal emission wavelengths. In some cases, each oligonucleotide probe comprises an identical fluorophore. In some cases, each oligonucleotide probe comprises a different fluorophore, where each fluorophore is capable of being detected in a single optical channel. In some cases, each oligonucleotide probe comprises a different fluorophore, where each fluorophore is capable of being detected in at least one optical channel.

A probe may correspond to a region of a target nucleic acid. For example, a probe may have complementarity and/or homology to a region of a target nucleic acid. A probe may comprise a region which is complementary or homologous to a region of a target nucleic acid. A probe corresponding to a region of a target nucleic acid may be capable of binding to the region of the target nucleic acid under appropriate conditions (e.g., temperature conditions, buffer conditions. etc.). For example, a probe may be capable of binding to a region of a target nucleic acid under conditions appropriate for polymerase chain reaction. An oligonucleotide probe may have less than 50%, 40%, 30%, 20%, 10%, 5%, or 1% complementarity to any member of a plurality of target nucleic acids. An oligonucleotide probe may have no complementarity to any member of the plurality of target nucleic acids. An oligonucleotide probe may hybridize specifically to a target nucleic, or derivative thereof, but not to non-target nucleic acid, or derivative thereof. For example, a target nucleic acid and a non-target a nucleic acid may be different at a divergence location comprising at least one nucleotide, and the probe may be configured to hybridize to the target and not hybridize to said divergence location comprising at least one nucleotide, or complement thereof. For example, a target nucleic acid and a non-target a nucleic acid may be different at a divergence location comprising at least one nucleotide, and the probe may be configured to hybridize to the target-specific tail segment and not hybridize to said divergence location comprising at least one nucleotide, or complement thereof.

In some aspects, an oligonucleotide probe may comprise a non-target-hybridizing sequence. A non-target-hybridizing sequence may be a sequence which is not complementary to any region of a target nucleic acid sequence. An oligonucleotide probe comprising a non-target-hybridizing sequence may be a hairpin detection probe. An oligonucleotide probe comprising a non-target-hybridizing sequence may be a molecular beacon probe. Examples of molecular beacon probes are provided in, for example, U.S. Pat. No. 7,671,184, incorporated herein by reference in its entirety. An oligonucleotide probe comprising a non-target-hybridizing sequence may be a molecular torch. Examples of molecular torches are provided in, for example, U.S. Pat. No. 6,534,274, incorporated herein by reference in its entirety.

Probe may have a wide variety of sequences that may bind or be configured to hybridize at a variety of region. A probe may be complementary or homologous to a target nucleic acid. A probe may be complementary or homologous only a target nucleic acid. A probe may be complementary or homologous to a portion of a target nucleic acid and a portion of a universal tail. A probe may be complementary or homologous to a portion of a universal tail. A probe may be complementary or homologous to only a portion of a universal tail. A probe may be complementary or homologous to a target-specific tail segment. A probe may be complementary or homologous to a universal probe binding motif. A probe may be configured to bind or hybridize to a target nucleic acid. A probe may be configured to bind or hybridize only a target nucleic acid. A probe may be configured to bind or hybridize to a portion of a target nucleic acid and a portion of a universal tail. A probe may be configured to bind or hybridize to a portion of a universal tail. A probe may be configured to bind or hybridize to only a portion of a universal tail. A probe may be configured to bind or hybridize to a target-specific tail segment. A probe may be configured to bind or hybridize to a universal probe binding motif. A probe may be configured to bind or hybridize to any combination of a portion of the universal tail, target specific tail segment, or target nucleic acid.

A probe may be provided at a specific concentration. In some cases, a second nucleic acid probe is provided at a concentration of at least about 2×, about 3×, about 4×, about 5×, about 6×, about 7×, about 8×, or more. In some cases, a second nucleic acid probe is provided at a concentration of at most about 8×, about 7×, about 6×, about 5×, about 4×, about 3×, or about 2×. In some cases, a second nucleic acid probe is provided at a concentration of about 2×, about 3×, about 4×, about 5×, about 6×, about 7×, or about 8×. X may be a concentration of a nucleic acid probe provided in the disclosed methods. In some cases, X is at least 50 nM, 100 nM, 150 nM, 200 nM, 250 nM, 300 nM, 350 nM, 400 nM, 450 nM, 500 nM, or greater. In some cases, X is at most 500 nM, 450 nM, 400 nM, 350 nM, 300 nM, 250 nM, 200 nM, 150 nM, 100 nM, or 50 nM. X may be any concentration of a nucleic acid probe which is capable of being partitioned.

An oligonucleotide probe may comprise a detectable label. A detectable label may be a chemiluminescent label. A detectable label may comprise a chemiluminescent label. A detectable label may comprise a fluorescent label. A detectable label may comprise a fluorophore. A fluorophore may be, for example, FAM, TET, HEX, JOE, Cy3, or Cy5. A fluorophore may be FAM. Each oligonucleotide probe used in the methods and assays of the presence disclosure may comprise at least one fluorophore. In some aspects, one or more oligonucleotide probes used in a single reaction may comprise the same fluorophore. In some aspects, one or more oligonucleotide probes used in a single reaction may comprise the dissimilar fluorophores. Each probe may, when excited and contacted with its corresponding target nucleic acid, generate a fluorescent signal. A single aggregate signal may comprise a plurality of signals may be generated from one or more probes.

In some aspects, an oligonucleotide probe may further comprise one or more quenchers. A quencher may inhibit signal generation from a fluorophore. A quencher may be, for example, TAMRA, BHQ-1, BHQ-2, or Dabcy. A quencher may be BHQ-1. A quencher may be BHQ-2.

Generally, a probe lacks a 3′ hydroxyl, and therefore is not extendable by the DNA polymerase. TaqMan® (ThermoFisher Scientific) chemistry is a common reporter probe used for multiplex real time PCR (Holland et al. 1991). The TaqMan® oligonucleotide probe is covalently modified with a fluorophore and a quenching tag (i.e., quencher). In this configuration the fluorescence generated by the fluorophore is quenched and is not detected by the real time PCR instrument. When the target of interest is present, the probe oligonucleotide base anneals with the amplified target. When bound, the TaqMan® oligonucleotide probe is digested by the 5′ to 3′ exonuclease activity of the Taq polymerase—thereby physically separating the fluorophore from the quencher and liberating signal for detection by the real time PCR instrument.

### Signal Generation

Thermal cycling may be performed such that one or more oligonucleotide probes are degraded by a nucleic acid enzyme. An oligonucleotide probe may be degraded by the exonuclease activity of a nucleic acid enzyme. An oligonucleotide probe may generate a signal upon degradation. In some cases, an oligonucleotide probe may generate a signal only if at least one member of a plurality of target nucleic acids is present in a mixture.

A reaction may generate one or more signals. A reaction may generate a cumulative intensity signal comprising a sum aggregate of multiple signals. A signal may be a chemiluminescent signal. A signal may be a fluorescent signal. A signal may be generated by an oligonucleotide probe. A sum signal may be generated by at least one oligonucleotide probe. For example, excitation of a hybridization probe comprising a luminescent signal tag may generate a signal. A signal may be generated by a fluorophore. A fluorophore may generate a signal upon release from a hybridization probe. A reaction may comprise excitation of a fluorophore. A reaction may comprise signal detection. A reaction may comprise detecting emission from a fluorophore.

A signal may be a fluorescent signal. A signal may correspond to a fluorescence intensity level. Each signal measured in the methods of the present disclosure may have a distinct fluorescence intensity value, thereby corresponding to the presence of a unique combination of target nucleic acids. A signal may be generated by one or more oligonucleotide probes. The number of signals generated in an assay may correspond to the number of oligonucleotide probes and target nucleic acids present.

N may be a number of signals detected in a single optical channel in an assay of the present disclosure. N may be at least 2, 3, 4, 5, 6, 7, 8, 9, 10, 11, 12, 13, 14, 15, 20, 25, 30, 40, 50 or more. N may be at most 50, 40, 30, 24, 20, 15, 14, 13, 12, 11, 10, 9, 8, 7, 6, 5, 4, 3, or 2. N may be 2, 3, 4, 5, 6, 7, 8, 9, 10, 11, 12, 13, 14, 15, 20, 25, 30, 40, or 50.

As will be recognized and is described elsewhere herein, sets of signals may be generated in multiple different optical channels, where each set of signals is detected in a single optical channel, thereby significantly increasing the number of target nucleic acids that can be measured in a single reaction. In some cases, two sets of signals are detected in a single reaction. Each set of signals detected in a reaction may comprise the same number of signals, or different numbers of signals.

In some cases, a signal may be generated simultaneous with hybridization of an oligonucleotide probe to a region of a nucleic acid. For example, an oligonucleotide probe (e.g., a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme.

In cases where an oligonucleotide probe comprises a signal tag, the oligonucleotide probe may be degraded when bound to a region of a primer, thereby generating a signal. For example, an oligonucleotide probe (e.g., a TaqMan® probe) may generate a signal following hybridization of the oligonucleotide probe to a nucleic acid and subsequent degradation by a polymerase (e.g., during amplification, such as PCR amplification). An oligonucleotide probe may be degraded by the exonuclease activity of a nucleic acid enzyme.

An oligonucleotide probe may comprise a quencher and a fluorophore, such that the quencher is released upon degradation of an oligonucleotide probe, thereby generating a fluorescent signal. Thermal cycling may be used to generate one or more signals. Thermal cycling may generate at least one, two, three, four, five, six, seven, eight, nine, ten signals, or more. Thermal cycling may generate at most ten, nine, eight, seven, six, five, four, three, two or one signal.

Multiple signals may be of the same type or of different types. Signals of different types may be fluorescent signals with different fluorescent wavelengths. Signals of different types may be generated by detectable labels comprising different fluorophores. Signals of the same type may be of different intensities (e.g., different intensities of the same fluorescent wavelength). Signals of the same type may be signals detectable in the same color channel. Signals of the same type may be generated by detectable labels comprising the same fluorophore. Detectable labels comprising the same fluorophore may generate different signals by nature of being at different concentrations, thereby generating different intensities of the same signal type.

The methods presented in this disclosure may be used with any quantifiable signal. In some cases, this disclosure provides methods to quantify targets using a single component of a signal (e.g., intensity). For example, an analysis may rely on a multiplicity of signal intensity without consideration of color. Although fluorescent probes have been used to illustrate this principle, the disclosed methods are equally applicable to any other method providing a quantifiable signal, including an electrochemical signal and a chemiluminescent signal.

The signal is described is not meant to be limiting, and one of ordinary skill in the art will readily recognize that the principles applicable to the measurement of a fluorescent signal are also applicable to other signals. For example, the methods presented in this disclosure may also utilize the measurement of a signal in at least two dimensions (e.g., color and intensity). In some cases, a quantifiable signal has both a frequency (wavelength) and an amplitude (intensity). A signal may be an electromagnetic signal. An electromagnetic signal may be a sound, a radio signal, a microwave signal, an infrared signal, a visible light signal, an ultraviolet light signal, an x-ray signal, or a gamma-ray signal. The wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths. Intensity may be measured with a photodetector. A range of wavelengths may be referred to as a “channel,” “color channel,” or “optical channel.”

### Detection

The presence or absence of one or more signals may be detected. The signals may be detected in single channel or multiple channels. The signals may be detected in a single channel detector or a multichannel detector. The signal may be detected by a fluorimeter. The signal may be detected by a real time PCR instrument. One signal may be detected, or multiple signals may be detected. Multiple signals may be detected simultaneously. Alternatively, multiple signals may be detected sequentially. The presence of a signal may be correlated to the presence of a target nucleic acid. The presence of least one, two, three, four, five, six, seven, eight, nine, ten, or more signals may be correlated with the presence of at least one of at least one, two, three, four, five, six, seven, eight, nine, ten, or more target nucleic acids. The absence of a signal may be correlated with the absence of corresponding target nucleic acids. The absence of least one, two, three, four, five, six, seven, eight, nine, ten, or more signals may be correlated with the absence of each of at least one two, three, four, five, six, seven, eight, nine, ten, or more target nucleic acid molecules.

### Identifying at Least One Target Nucleic Acid

Described herein, in some aspects, is a method of identifying at least one target nucleic acid in a sample. First, a sample may be provided comprising a plurality of nucleic acid molecules. Next a mixture may be formed by adding a plurality of oligonucleotide probes. The plurality of nucleic acid molecules may be derived from, and/or may correspond with the at least one target nucleic acid in the sample. The plurality of oligonucleotide probes may each correspond to a different region of the target nucleic acid. The mixture may further comprise other reagents (e.g., amplification reagents) including, for example, primers, dNTPs, a nucleic acid enzyme (e.g., a polymerase), buffers, and salts (e.g., Ca²⁺, Mg²⁺, etc.). A pair of primers may be configured to amplify a nucleic acid sequence of a length of at least 50, at least 75, at least 100, at least 125, at least 150, at least 175, at least 200, at least 225, at least 250, at least 275, or at least 300 base pairs (bp), or more. Next, the mixture may be amplified, thereby generating a plurality of signals. The plurality of signals may be detectable in one color channel. The plurality of signals may be detectable in multiple color channels.

The plurality of signals may be generated by one or more of the plurality of probes from the mixture. The plurality of signals may be generated by nucleic acid amplification (e.g., PCR) of the plurality of nucleic acid molecules. Nucleic acid amplification may degrade the pluralities of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

Quantifying the at least one target nucleic acid may comprise determining a ratio of the target nucleic acid to a reference standard (e.g., the quantity of the target nucleic acid relative to a known reference). Quantifying the at least one target nucleic acid may comprise determining an absolute quantity of the target nucleic acid in the sample. Quantifying the at least one target nucleic acid may comprise determining a relative quantity of target nucleic acid to a reference.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.).

### Determining a Ratio

Described herein, in some aspects, is a method of determining the presence or absence of a target relative to a second target nucleic acid in a sample. First, a sample may be provided comprising a first plurality of nucleic acid molecules and a second plurality of nucleic acid molecules. The first plurality of nucleic acid molecules may be derived from, and/or may correspond with, the first target nucleic acid in the sample. The second plurality of nucleic acid molecules may be derived from, and/or may correspond with, the second target nucleic acid in the sample. In addition to the first and second pluralities of nucleic acid molecules, other reagents (e.g., amplification reagents) may be provided in the sample, including, for example, primers, oligonucleotide probes, dNTPs, a nucleic acid enzyme (e.g., a polymerase), and salts (e.g., Ca²⁺, Mg²⁺, etc.). Next, the first plurality of nucleic acid molecules and the second plurality of nucleic acid molecules may be amplified, thereby generating a plurality of signals. The plurality of signals may be detectable in one color channel. The plurality of signals may be detectable in multiple color channels. Next, the plurality of signals may be detected. The plurality of signals may be detected in a single-color channel. The plurality of signals may be detected in multiple color channels. Next, based on the detecting, a ratio may be determined which is representative of a quantity of the first target nucleic acid relative to a quantity of the second target nucleic acid in the sample.

### Kits

The present disclosure also provides kits for analysis of the determination of the presence of a target in the presence of a non-target. The kits may also be used for multiplexed analysis. Kits may comprise one or more oligonucleotide probe as described herein. Oligonucleotide probes may be lyophilized. Different oligonucleotide probes may be present at different concentrations in a kit. Oligonucleotide probes may comprise a fluorophore and/or one or more quenchers.

Kits may comprise one or more sets of primers (or “amplification oligomers”) as described herein. A set of primers may comprise paired primers. Paired primers may comprise a forward primer and a reverse primer. A set of primers may be configured to amplify a nucleic acid sequence corresponding to particular targets. For example, a forward primer may be configured to hybridize to a first area (e.g., a 3′ end) of a nucleic acid sequence, and a reverse primer may be configured to hybridize to a second area (e.g., a 5′ end) of the nucleic acid sequence, thereby being configured to amplify the nucleic acid sequence. Different sets of primers may be configured to amplify nucleic acid sequences. In one example, a first set of primers may be configured to amplify a first nucleic acid sequence, and a second set of primers may be configured to amplify a second nucleic acid sequence. Primers configured to amplify nucleic acid molecules may be used in performing the disclosed methods. In some cases, all of the primers in a kit are lyophilized.

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. A nucleic acid enzyme may be capable of degrading a nucleic acid comprising a non-natural nucleotide. A nucleic acid enzyme may be an endonuclease V such as, for example, an E. coli endonuclease V. A nucleic acid enzyme may be a polymerase (e.g., a DNA polymerase). A polymerase may be Taq polymerase or a variant thereof. A nucleic acid enzyme may be capable, under appropriate conditions, of degrading an oligonucleotide probe. A nucleic acid enzyme may be capable, under appropriate conditions, of releasing a quencher from an oligonucleotide probe. Kits may comprise instructions for using any of the foregoing in the methods described herein.

Kits provided herein may be useful in, for example, calculating at least first and second sums, each being a sum of multiple target signals corresponding with a first and second target nucleic acids

### Systems

The present disclosure also provides system for the determination of the presence of a target nucleic acid in the presence of a non-target nucleic acid. The systems may comprise additional apparatuses to perform the steps of the methods described elsewhere herein. For example, the system may comprise a detector to detect signals. The system may comprise a thermocycler or other apparatus to subject the target nucleic acids, mixtures, or samples to conditions, such as amplification conditions appropriate to amplify the target nucleic acid with an amplification reaction. The system may also comprise apparatus to hold the mixtures, nucleic acids and samples.

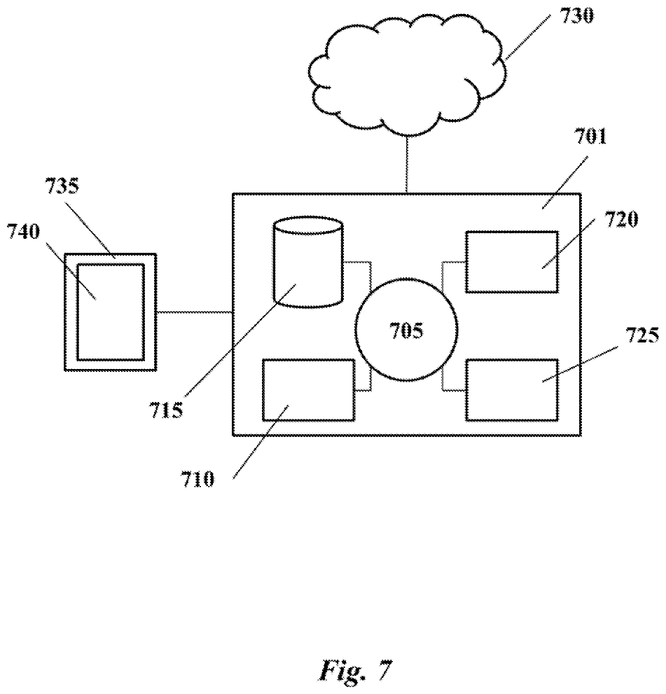

The present disclosure provides computer control systems that are programmed to implement methods of the disclosure. FIG. 7 shows a computer system 701 that is programmed or otherwise configured to perform methods, detect signals, subject mixtures to appropriate conditions. The computer system 701 can regulate various aspects of the present disclosure, such as, for example, control the temperature and time of thermocycling, detect a signal in a particular channel, filer out wavelength in particular channel. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, prompts to continue to the next steps of method, graphs of signal curves for plotting and manipulating. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, determine the presence of a signal based on a threshold or reduce noise of the signal.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for determining the presence or absence of a target nucleic acid in the presence of a non-target nucleic acid, said method comprising:
(A) providing a sample comprising, or potentially comprising, said target nucleic acid and said non-target nucleic acid;
(B) forming a mixture comprising:
i. said sample;
ii. a forward primer comprising a first region configured to hybridize to said target nucleic acid under amplification conditions and configured to not hybridize to said non-target nucleic acid under said amplification conditions, and a second region configured to not hybridize to said target nucleic acid under said amplification conditions; and
iii. a signal generating nucleic acid probe, wherein said signal generating nucleic acid probe anneals to said second region, or regions complementary thereto, when subjected to said amplification conditions;
(C) subjecting said mixture to said amplification conditions, said amplification conditions appropriate to amplify said target nucleic acid with an amplification reaction, thereby amplifying said target nucleic acid such that said signal generating nucleic acid probe is degraded and a signal is generated if said target nucleic acid is present in said mixture; and
(D) detecting the presence or absence of said signal, thereby determining the presence or absence of said target nucleic acid in the presence of said non-target nucleic acid.

2. The method of claim 1, wherein said target nucleic acid is an allele.

3. The method of claim 1, wherein said target nucleic acid is a mutant sequence and said non-target nucleic is a wild type sequence.

4. The method of claim 1, wherein the sequence of said target nucleic acid and the sequence of said non-target nucleic acid is different at least one nucleotide.

5. The method of claim 1, wherein the sequence of said target nucleic acid and the sequence of said non-target nucleic acid is different by only one nucleotide.

6. The method of claim 1, wherein the sequence of said target nucleic acid and the sequence of said non target nucleic acid is different by no more than five nucleotides.

7. The method of claim 1, wherein said forward primer is specific to and selectively amplifies only said target nucleic acid.

8. The method of claim 1, wherein said mixture does not employ the use of a nucleic acid or peptide blocking agents.

9. The method of claim 1, wherein said mixture further comprising a reverse primer.

10. The method of any of claims 1-9, wherein said forward primer is configured to hybridize to a first area of a nucleic acid sequence of said target nucleic acid, and said reverse primer is configured to hybridize to a second area of the nucleic acid sequence of said target nucleic acid, thereby being configured to amplify the nucleic acid sequence under conditions sufficient for nucleic acid amplification.

11. The method of claim 10, wherein said first area comprises a 3′ end of said nucleic acid sequence.

12. The method of claim 10, wherein said second area comprises a 5′ end of said nucleic acid sequence.

13. The method of claim 9, wherein said reverse primer comprises a sequence with complementarity or is homologous to a sequence on both target nucleic acid and non-target nucleic acid.

14. The method of claim 9, wherein said reverse primer is a locus specific primer.

15. The method of claim 9, wherein said reverse primer is a universal primer.

16. The method of claim 9, wherein said reverse primer is configured such that upon said thermally cycling said signal generating nucleic acid probe is digested.

17. The method of claim 1, wherein said mixture further comprises a nucleic acid enzyme.

18. The method of claim 1, wherein said amplification reaction is a polymerase chain reaction (PCR).

19. The method of claim 18, wherein said PCR is a quantitative polymerase chain reaction (qPCR).

20. The method of claim 1, wherein said amplification conditions comprises: a dNTP, a salt, a buffer, or a combination thereof.

21. The method of claim 1, wherein said signal generating nucleic acid probe further comprises a signal tag.

22. The method of claim 21, wherein said signal tag generates said signal.

23. The method of claim 22, wherein said signal tag generates said signal upon degradation of said cleavable, signal generating nucleic acid probe by the 5′ to 3′ exonuclease activity of said nucleic acid enzyme, thereby liberating said signal for detection by a real time PCR instrument.

24. The method of any of claims 1-23, wherein said amplification conditions comprise thermal cycling and each thermal cycle is performed at an annealing temperature appropriate for annealing said forward primer to said target nucleic acid.

25. The method of claim 1, wherein said second region comprises:
(A) a target-specific tail segment; and
(B) a universal tail segment.

26. The method of claim 25, wherein said first region is positioned on the 3′ end of forward primer.

27. The method of claim 25, wherein said universal tail segment is located on the 5′ end of said forward primer.

28. The method of claim 25, wherein said target-specific tail segment is flanked on the 3′ by said target-specific segment, and flanked on the 5′ end by said universal tail segment on said forward primer.

29. The method of claim 25, wherein a plurality of forward primers is used, wherein each second region of said forward primers comprises identical nucleotide sequences.

30. The method of claim 25, wherein a plurality of forward primers is used, wherein each second region of said forward primers comprises dissimilar nucleotide sequences.

31. The method of claim 25, wherein a plurality of forward primers is used, wherein each second region of said forward primers comprises unique nucleotide sequences.

32. The method of claim 1, wherein said signal generating nucleic acid probe comprises sequence complementary or homologous to said forward primer.

33. The method of claim 1, wherein said signal generating nucleic acid probe is a target-specific probe.

34. The method of claim 25, wherein said signal generating nucleic acid probe is a target-specific probe comprising:
(A) a sequence complementary or homologous to said 3′ end of said universal tail;
(B) a sequence complementary or homologous to said entire target-specific tail; and
(C) a sequence complementary or homologous to a portion of said first region.

35. The method of claim 25, wherein said signal generating nucleic acid probe is a target-specific probe comprising:
(A) a sequence complementary or homologous to said 3′ end of said universal tail; and
(B) a sequence complementary or homologous to a portion of the target-specific tail.

36. The method of claim 1, wherein said probe binds to said target nucleic acid in an amplicon generated by second strand synthesis initiated in said amplification reaction.

37. The method of claim 1, wherein the mixture further comprises a second nucleic acid primer.

38. The method of claim 37, wherein said second nucleic acid primer is a target specific primer.

39. The method of claim 37, wherein said second nucleic acid primer is configured to hybridize with said target nucleic acid or a derivative thereof and not to hybridize with said non-target nucleic or derivative thereof.

40. The method of claim 37, wherein the sequence of said target nucleic acid and the sequence of said non-target nucleic acid are different at least one nucleotide.

41. The method of claim 37, wherein the sequence of said target nucleic acid and the sequence of said non-target nucleic acid are different by only one nucleotide.

42. The method of claim 37, wherein the sequence of said target nucleic acid and the sequence of said non-target nucleic acid are different by no more than five nucleotides.

43. The method of claim 38, wherein said target specific primer is homologous or complementary to a sequence of said target nucleic acid.

44. The method of claim 37, wherein said second nucleic acid primer is a universal primer.

45. The method of claim 44, wherein said universal primer is complementary or homologous to a sequence of said second region.

46. The method of claim 37, wherein said second nucleic acid primer is configured to digest said cleavable, signal generating probe upon thermocycling.

47. The method of claim 1, wherein said signal generating nucleic acid probe comprises a sequence homologous or complementary to said target nucleic acid.

48. The method of claim 1, wherein said signal generating probe is configured to anneal to said second region and said first region.

49. The method of claim 1, wherein said signal generating nucleic acid probe is configured to hybridize to said second region or said first region.

50. The method of claim 1, wherein said signal generating nucleic acid probe is configured to hybridize to only said first region.

51. The method of claim 1, wherein said signal generating nucleic acid probe is configured to hybridize to only said second region.

52. The method of claim 25, wherein said signal generating nucleic acid probe is configured to hybridize to said target-specific tail segment.

53. The method of claim 25, wherein said signal generating nucleic acid probe is configured to hybridize to said target-specific tail segment and not hybridize to a sequence, or complement thereof, of said target nucleic acid.

54. The method of claim 25, wherein said target nucleic acid and said non-target a nucleic acid are different at a divergence location comprising at least one nucleotide, and said signal generating nucleic acid probe is configured to hybridize to said target-specific tail segment and not hybridize to said divergence location comprising at least one nucleotide, or complement thereof.

55. The method of claim 1, wherein said second region comprises:
(A) a universal probe binding motif; and
(B) a universal primer binding motif.

56. The method of claim 55, wherein said signal generating probe is configured to bind said universal probe binding motif.

57. The method of claim 55, wherein said mixture further comprise a universal primer configured to bind said universal primer binding motif.

58. The method of claim 55, wherein said target-specific segment is positioned on the 3′ end of said forward primer.

59. The method of claim 55, wherein said universal primer binding motif is located on the 5′ end of said universal tail primer.

60. The method of claim 55, wherein said universal probe binding motif is flanked by the 5′ end of said first region, and the 3′ end of said universal primer binding motif.

61. The method of claim 55, wherein a plurality of forward primers are used, each universal primer binding motif comprises identical nucleotide sequences.

62. The method of claim 55, wherein a plurality of forward primers are used, each universal primer binding motifs comprises dissimilar nucleotide sequences.

63. The method of claim 55, wherein said second region comprises a second universal probe binding motif.

64. The method of claim 55, wherein said mixture further comprises a probe with a sequence complementary or homologous to said second universal probe binding motif.

65. The method of claim 63, wherein said universal probe binding motif and said second universal probe binding motif comprise the same sequence.

66. The method of claim 63, wherein said universal probe binding motif and said second universal probe binding motif comprise a different sequence.

67. The method of claim 63, wherein said universal probe binding motif and said second universal probe binding motif are configured to bind the same probe sequence.

68. The method of claim 63, wherein said universal probe binding motif and said second universal probe binding motif are configured to bind a different probe sequence.

69. The method of claim 63, wherein said second universal probe binding motif is adjacent to said universal binding motif and said second universal probe binding motif and said universal binding motif are on the 3′ end of said first region and are on the 5′ of said universal primer binding motif.

70. The method of claim 63, wherein a plurality of said forward primers are used, each universal primer binding motif comprises identical nucleotide sequences.

71. The method of claim 63, wherein said plurality of said forward primers are used, each universal primer binding motif comprises dissimilar nucleotide sequences.

72. The method of claim 63, wherein said plurality of forward primers is used, each universal probe binding motif comprises a unique nucleotide sequence.

73. A method for determining the presence or absence of a target nucleic acid in the presence of a non-target nucleic acid, said method comprising:
(A) providing a sample comprising said target nucleic acid;
(B) forming a mixture comprising:
i. said sample;
ii. a non-extendible nucleic acid primer, wherein said non-extendible nucleic acid primer comprises a first region configured to hybridize to said target nucleic acid and configured not to bind to said non-target nucleic acid, and a second region configured to not hybridize to said target nucleic acid, and wherein said non-extendible nucleic acid primer comprises a target-specific RNA base, DNA base, or unnatural base, configured to, when said first region is hybridized to said target nucleic acid, form a base hybrid pair at a target base of said target nucleic acid and not form said base hybrid pair at a corresponding base in the non-target nucleic acid; and
iii. a signal generating nucleic acid probe;
(C) subjecting said mixture to cleavage conditions such that nucleic acids comprising said base hybrid pair are cleaved such that said non-extendible nucleic acid is cleaved, thereby converting said non-extendible nucleic acid primer to an extendible primer;
(D) subjecting said mixture to said amplification conditions, said amplification conditions appropriate to amplify said target nucleic acid with an amplification reaction, such that said signal generating nucleic acid probe is degraded and a signal is generated if said target nucleic acid is present in said mixture; and
(E) detecting the presence or absence of said signal, thereby determining the presence or absence of said target nucleic acid in the presence of said non-target nucleic acid.

74. The method of claim 73, wherein said amplification reaction comprises a reverse transcriptase polymerase chain reaction (RT-PCR) or a polymerase chain reaction (PCR).

75. The method of claim 73, wherein said target-specific RNA base, DNA base, or unnatural base of said non-extendible nucleic acid primer is at a location complementary to said target nucleic acid.

76. The method of claim 73, wherein said target-specific RNA base, DNA base, or unnatural base facilitates the selective amplification of said target nucleic acid.

77. The method of claim 73, wherein said base hybrid pair is a DNA:RNA pair, a DNA:unnatural base, or a RNA:unnatural base pair.

78. The method of claim 73, wherein in c), said non-extendible nucleic acid primer is cleaved at a location adjacent to or at said base hybrid pair.

79. The method of claim 73, wherein said cleavage conditions comprise an additional reagent or enzyme for cleaving said first non-extendible nucleic acid primer.

80. The method of claim 79, wherein said additional enzyme is ribonuclease H.

81. The method of claim 73, wherein said target nucleic acid is an allele.

82. The method of claim 73, wherein said target nucleic acid is a mutant sequence and said non-target nucleic is a wild type sequence.

83. The method of claim 73, wherein the sequence of said target nucleic acid and the sequence of said non-target nucleic acid is different at least one nucleotide.

84. The method of claim 73, wherein the sequence of said target nucleic acid and the sequence of said non-target nucleic acid is different by only one nucleotide.

85. The method of claim 73, wherein the sequence of said target nucleic acid and the sequence of said non target nucleic acid is different by no more than five nucleotides.

86. The method of claim 73, wherein said extendible primer is specific to and selectively amplifies only said target nucleic acid.

87. The method of claim 73, wherein said mixture does not employ the use of a nucleic acid or peptide blocking agents.

88. The method of claim 73, wherein said mixture further comprising a reverse primer.

89. The method of any of claims 73-88, wherein said extendible primer is configured to hybridize to a first area of a nucleic acid sequence of said target nucleic acid, and said reverse primer is configured to hybridize to a second area of the nucleic acid sequence of said target nucleic acid, thereby being configured to amplify the nucleic acid sequence under conditions sufficient for nucleic acid amplification.

90. The method of claim 89, wherein said first area comprises a 3′ end of said nucleic acid sequence.

91. The method of claim 89, wherein said second area comprises a 5′ end of said nucleic acid sequence.

92. The method of claim 88, wherein said reverse primer comprises a sequence with complementarity or is homologous to a sequence on both target nucleic acid and non-target nucleic acid.

93. The method of claim 88, wherein said reverse primer is a locus specific primer.

94. The method of claim 88, wherein said reverse primer is a universal primer.

95. The method of claim 88, wherein said reverse primer is configured such that upon said thermally cycling said signal generating nucleic acid probe is digested.

96. The method of claim 73, wherein said mixture further comprises a nucleic acid enzyme.

97. The method of claim 73, wherein said amplification reaction is a polymerase chain reaction (PCR).

98. The method of claim 97, wherein said PCR is a quantitative polymerase chain reaction (qPCR).

99. The method of claim 73, wherein said amplification conditions comprises: a dNTP, a salt, a buffer, or a combination thereof.

100. The method of claim 73, wherein said signal generating nucleic acid probe further comprises a signal tag.

101. The method of claim 100, wherein said signal tag generates said signal.

102. The method of claim 101, wherein said signal tag generates said signal upon degradation of said signal generating nucleic acid probe by the 5′ to 3′ exonuclease activity of said nucleic acid enzyme, thereby liberating said signal for detection by a real time PCR instrument.

103. The method of any of claims 73-102, wherein said amplification conditions comprise thermal cycling and each thermal cycle is performed at an annealing temperature appropriate for annealing said extendible primer to said target nucleic acid.

104. The method of claim 73, wherein said second region comprises:
(A) a target-specific tail segment; and
(B) a universal tail segment.

105. The method of claim 73, wherein said first region is positioned on the 3′ end of extendible primer.

106. The method of claim 104, wherein said universal tail segment is located on the 5′ end of said extendible primer.

107. The method of claim 104, wherein said target-specific tail segment is flanked on the 3′ by said target-specific segment, and flanked on the 5′ end by said universal tail segment on said non-extendible primer.

108. The method of claim 104, wherein a plurality of non-extendible primers is used, wherein each second region of said non-extendible primers comprises identical nucleotide sequences.

109. The method of claim 104, wherein a plurality of non-extendible primers is used, wherein each second region of said non-extendible primers comprises dissimilar nucleotide sequences.

110. The method of claim 104, wherein a plurality of non-extendible primers is used, wherein each second region of said non-extendible primers comprises unique nucleotide sequences.

111. The method of claim 73, wherein said signal generating nucleic acid probe comprises sequence complementary or homologous to said non-extendible primer.

112. The method of claim 73, wherein said signal generating nucleic acid probe is a target-specific probe.

113. The method of claim 104, wherein said signal generating nucleic acid probe is a target-specific probe comprising:
(A) a sequence complementary or homologous to said 3′ end of said universal tail;
(B) a sequence complementary or homologous to said entire target-specific tail; and
(C) a sequence complementary or homologous to a portion of said first region.

114. The method of claim 104, wherein said signal generating nucleic acid probe is a target-specific probe comprising:
(A) a sequence complementary or homologous to said 3′ end of said universal tail; and
(B) a sequence complementary or homologous to a portion of the target-specific tail.

115. The method of claim 73, wherein said probe binds to said target nucleic acid in an amplicon generated by second strand synthesis initiated in said amplification reaction.

116. The method of claim 73, wherein the mixture further comprises a second nucleic acid primer.

117. The method of claim 116, wherein said second nucleic acid primer is a target specific primer.

118. The method of claim 116, wherein said second nucleic acid primer is configured to hybridize with said target nucleic acid or a derivative thereof and not to hybridize with said non-target nucleic or derivative thereof.

119. The method of claim 116, wherein the sequence of said target nucleic acid and the sequence of said non-target nucleic acid are different at least one nucleotide.

120. The method of claim 116, wherein the sequence of said target nucleic acid and the sequence of said non-target nucleic acid are different by only one nucleotide.

121. The method of claim 116, wherein the sequence of said target nucleic acid and the sequence of said non-target nucleic acid are different by no more than five nucleotides.

122. The method of claim 117, wherein said target specific primer is homologous or complementary to a sequence of said target nucleic acid.

123. The method of claim 116, wherein said second nucleic acid primer is a universal primer.

124. The method of claim 123, wherein said universal primer is complementary or homologous to a sequence of said second region.

125. The method of claim 116, wherein said second nucleic acid primer is configured to digest said cleavable, signal generating probe upon thermocycling.

126. The method of claim 73, wherein said signal generating probe comprises a sequence homologous or complementary to said target nucleic acid.

127. The method of claim 73, wherein said signal generating probe is configured to anneal to said second region and said first region.

128. The method of claim 73, wherein said signal generating nucleic acid probe is configured to hybridize to said second region or said first region.

129. The method of claim 73, wherein said signal generating nucleic acid probe is configured to hybridize to only said first region.

130. The method of claim 73, wherein said signal generating nucleic acid probe is configured to hybridize to only said second region.

131. The method of claim 104, wherein said signal generating nucleic acid probe is configured to hybridize to said target-specific tail segment.

132. The method of claim 104, wherein said signal generating nucleic acid probe is configured to hybridize to said target-specific tail segment and not hybridize to a sequence, or complement thereof, of said target nucleic acid.

133. The method of claim 104, wherein said target nucleic acid and said non-target a nucleic acid are different at a divergence location comprising at least one nucleotide, and said signal generating nucleic acid probe is configured to hybridize to said target-specific tail segment and not hybridize to said divergence location comprising at least one nucleotide, or complement thereof.

134. The method of claim 73, wherein said second region comprises:
(A) a universal probe binding motif; and
(B) a universal primer binding motif.

135. The method of claim 134, wherein said signal generating probe is configured to bind said universal probe binding motif.

136. The method of claim 134, wherein said mixture further comprise a universal primer configured to bind said universal primer binding motif.

137. The method of claim 134, wherein said target-specific segment is positioned on the 3′ end of said non-extendible primer.

138. The method of claim 134, wherein said universal primer binding motif is located on the 5′ end of said universal tail primer.

139. The method of claim 134, wherein said universal probe binding motif is flanked by the 5′ end of said first region, and the 3′ end of said universal primer binding motif.

140. The method of claim 134, wherein a plurality of non-extendible primers is used, each universal primer binding motif comprises identical nucleotide sequences.

141. The method of claim 134, wherein a plurality of non-extendible primers is used, each universal primer binding motifs comprises dissimilar nucleotide sequences.

142. The method of claim 134, wherein said second region comprises a second universal probe binding motif.

143. The method of claim 134, wherein said mixture further comprises a probe with a sequence complementary or homologous to said second universal probe binding motif.

144. The method of claim 143, wherein said universal probe binding motif and said second universal probe binding motif comprise the same sequence.

145. The method of claim 143, wherein said universal probe binding motif and said second universal probe binding motif comprise a different sequence.

146. The method of claim 143, wherein said universal probe binding motif and said second universal probe binding motif are configured to bind the same probe sequence.

147. The method of claim 143, wherein said universal probe binding motif and said second universal probe binding motif are configured to bind a different probe sequence.

148. The method of claim 143, wherein said second universal probe binding motif is adjacent to said universal binding motif and said second universal probe binding motif and said universal binding motif are on the 3′ end of said first region and are on the 5′ of said universal primer binding motif.

149. The method of claim 143, wherein a plurality of said non-extendible primers are used, each universal primer binding motif comprises identical nucleotide sequences.

150. The method of claim 143, wherein said plurality of said non-extendible primers are used, each universal primer binding motifs comprises dissimilar nucleotide sequences.

151. The method of claim 143, wherein said plurality of non-extendible primers is used, each of said at least two universal probe binding motifs comprises a unique nucleotide sequence.

152. A kit for use in determining the presence or absence of a target nucleic acid in the presence of a non-target nucleic acid, said kit comprising:
(A) a forward primer comprising a first region configured to hybridize to said target nucleic acid under amplification conditions and configured to not hybridize to said non-target nucleic acid under said amplification conditions, and a second region configured to not hybridize to said target nucleic acid under said amplification conditions;
(B) a signal generating nucleic acid probe, wherein said signal generating nucleic acid probe anneals to said second region, or regions complementary thereto, when subjected to said amplification conditions, and wherein said signal generating nucleic acid probe is configured, upon introduction of a target nucleic acid and exposure to appropriate conditions, to be degraded, and a signal is generated if said target nucleic acid is present in said mixture; and the presence or absence of said signal is configured to be detected; and
(C) instructions.

153. A system for determining the presence or absence of a target nucleic acid in the presence of a non-target nucleic acid, said system comprising:
(A) a reaction vessel configured to receive:
i. said sample;
ii. a forward primer comprising a first region configured to hybridize to said target nucleic acid under amplification conditions and configured to not hybridize to said non-target nucleic acid under said amplification conditions, and a second region configured to not hybridize to said target nucleic acid under said amplification conditions; and
iii. a signal generating nucleic acid probe, wherein said signal generating nucleic acid probe anneals to said second region, or regions complementary thereto, when subjected to said amplification conditions;
(B) a thermocycler configured to subject said mixture to said amplification conditions, said amplification conditions appropriate to amplify said target nucleic acid with an amplification reaction, thereby amplifying said target nucleic acid such that said signal generating nucleic acid probe is degraded and a signal is generated if said target nucleic acid is present in said mixture; and
(C) a detector configured to detect the presence or absence of said signal, thereby determining the presence or absence of said target nucleic acid in the presence of said non-target nucleic acid.

154. A system, comprising a controller comprising or capable of accessing, computer readable media comprising non-transitory computer-executable instructions which, when executed by at least one electronic processor perform a method comprising:
(A) providing a sample comprising, or potentially comprising, said target nucleic acid and said non-target nucleic acid;
(B) forming a mixture comprising:
i. said sample;
ii. a forward primer comprising a first region configured to hybridize to said target nucleic acid under amplification conditions and configured to not hybridize to said non-target nucleic acid under said amplification conditions, and a second region configured to not hybridize to said target nucleic acid under said amplification conditions; and
iii. a signal generating nucleic acid probe, wherein said signal generating nucleic acid probe anneals to said second region, or regions complementary thereto, when subjected to said amplification conditions;
(C) subjecting said mixture to said amplification conditions, said amplification conditions appropriate to amplify said target nucleic acid with an amplification reaction, thereby amplifying said target nucleic acid such that said signal generating nucleic acid probe is degraded and a signal is generated if said target nucleic acid is present in said mixture; and
(D) detecting the presence or absence of said signal, thereby determining the presence or absence of said target nucleic acid in the presence of said non-target nucleic acid.
